(19) United States
(12) Patent Application Publication
(10) Pub. No.: US 2025/0331984 A1
(43) Pub. Date: Oct. 30, 2025

(54) OUTER SKIRTS FOR EXPANDABLE PROSTHETIC HEART VALVES

(71) Applicant: EDWARDS LIFESCIENCES CORPORATION, Irvine, CA (US)

(72) Inventors: Michael G. Valdez, Riverside, CA (US); Tiana Tran, Westminster, CA (US)

(21) Appl. No.: 19/263,331

(22) Filed: Jul. 8, 2025

Related U.S. Application Data
(63) Continuation of application No. PCT/US2024/010362, filed on Jan. 4, 2024.
(60) Provisional application No. 63/479,478, filed on Jan. 11, 2023.

(51) Int. Cl. A61F 2/24

(52) U.S. Cl. CPC A61F 2/2418; A61F 2/2439; A61F 2220/0075; A61F 2250/0069

(57) ABSTRACT

Sealing members for prosthetic heart valves are disclosed. As one example, a prosthetic valve can include a radially expandable frame, a valvular structure disposed within the frame, and a sealing member disposed around an outer surface of the frame. The sealing member can have an inflow end, an outflow end, and a frame attachment region positioned axially between the inflow and outflow ends. The sealing member can include a radially inner layer and a radially outer layer, wherein the sealing member is coupled to the frame at the frame attachment region.

### CROSS-REFERENCE TO RELATED APPLICATION

This application is a continuation of PCT Patent Application No. PCT/US2024/010362 filed on Jan. 4, 2024, which application claims the benefit of U.S. Provisional Patent Application No. 63/479,478, filed Jan. 11, 2023, each of these applications being incorporated by reference herein in its entirety.

### FIELD

The present disclosure relates to expandable prosthetic valves, including outer skirts for prosthetic valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Percutaneous prosthetic valves (also known as transcatheter heart valves) typically have an outer skirt or sealing member that extends around the outer surface of the frame of the prosthetic valve. When the prosthetic valve is expanded within a native heart valve, the outer skirt contacts tissue of the surrounding native valve, thereby establishing a seal between the prosthetic valve and the surrounding tissue that prevents or reduces paravalvular leakage. Depending on the patient's anatomy, the native valve can have an irregular shape, such as due to the presence of calcium nodules, which can prevent the outer skirt from fully sealing against the surrounding tissue.

Accordingly, a need exists for improved skirt assemblies for preventing or minimizing paravalvular leakage.

### SUMMARY

Described herein are prosthetic heart valves, delivery apparatus, and methods for implanting prosthetic heart valves. The disclosed prosthetic heart valves, delivery apparatus, and methods can, for example, provide outer skirts that bulge or flex away from a frame of the prosthetic valve when the valve is in an expanded state and maintain a low profile when the valve is in a crimped state. The disclosed skirts can help to ensure that the prosthetic heart valve establishes a full seal against the native valve, such that paravalvular leakage is prevented or minimized. As such, the devices and methods disclosed herein can, among other things, overcome one or more of the deficiencies of typical prosthetic heart valves and their delivery apparatus.

A prosthetic heart valve can comprise a frame and a valvular structure coupled to the frame. In addition to these components, a prosthetic heart valve can further comprise one or more of the components disclosed herein.

In some examples, the prosthetic heart valve can comprise a sealing member configured to reduce paravalvular leakage.

In some examples, the sealing member is coupled to the frame at a frame attachment region located axially between an inflow end and an outflow end of the sealing member such that the inflow and outflow ends can move in a radial direction relative to the frame.

In some examples, the sealing member is coupled to the frame adjacent the inflow end of the sealing member such that the outflow end of the sealing member can move in a radial direction relative to the frame.

In some examples, the sealing member is coupled to the frame with a circumferentially extending stitch line.

In some examples, the sealing member comprises a plurality of axially extending pleats. In some examples, the pleats can extend from the inflow end to the outflow end of the sealing member. In some examples, the pleats can overlap each other in a circumferential direction.

In some examples, the sealing member comprises an inner layer and an outer layer.

In some examples, the sealing member comprises at least one spacer disposed between the inner layer and the outer layer. In some examples, the at least one spacer comprises a compressible material.

In some examples, the sealing member comprises a plurality of discrete segments. In some examples, one or more of the discrete segments can overlap an adjacent segment.

In some examples, the sealing member comprises an outer layer comprising a pile texture.

In some examples, the prosthetic valve can include a plurality of sealing members disposed around the frame.

In some examples, a prosthetic heart valve comprises one or more of the components recited in Examples 1-28.

In some examples, a prosthetic valve comprises a radially expandable frame; a valvular structure disposed within the frame; and a sealing member disposed around an outer surface of the frame, the sealing member having an inflow end, an outflow end, and a frame attachment region positioned axially between the inflow and outflow ends, wherein the sealing member comprises an inner layer and an outer layer, wherein the sealing member is coupled to the frame at the frame attachment region.

In some examples, a prosthetic valve comprises a radially expandable frame; a valvular structure disposed within the frame; and a sealing member disposed around an outer surface of the frame, the sealing member comprising at least one pleat portion having an inner layer and an outer layer, wherein the pleat portion includes a fixed end coupled to the frame and a free end that is moveable relative to the frame.

In some examples, a prosthetic valve comprises a radially expandable frame; a valvular structure disposed within the frame; and a sealing member disposed around an outer surface of the frame and comprising a plurality of axially-extending pleats.

In some examples, a prosthetic valve comprises a radially expandable frame having an inflow end portion and an outflow end portion; a valvular structure disposed within the frame; and a sealing member disposed around an outer surface of the frame, the sealing member comprising a fixed end coupled to the frame and a free end moveable relative to the frame, the sealing member configured to be stretched in an axial direction by a force applied in an axial direction to the free end.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a side view of a prosthetic heart valve, according to one example.

FIG. 2 is a side view of a frame of the prosthetic heart valve of FIG. 1.

FIG. 3 is a side view of an exemplary delivery apparatus configured to deliver and implant a radially expandable prosthetic heart valve at an implantation site.

FIG. 4 is a side view of an outer skirt coupled to a prosthetic heart valve, according to one example.

FIG. 5 is a partial cross-sectional view of the outer skirt of FIG. 4.

FIG. 6 is an end view of the prosthetic heart valve of FIG. 4.

FIG. 7 illustrates the prosthetic heart valve of FIG. 4 positioned within an aortic annulus of a heart.

FIG. 8 is a partial cross-sectional view of the outer skirt of FIG. 4 with a spacer included therein.

FIG. 9 is a partial cross-sectional view of the outer skirt of FIG. 4 with a sealing member having varied thickness, according to one example.

FIG. 10 is a side view of an outer skirt coupled to a prosthetic heart valve, according to one example.

FIG. 11 is a partial cross-sectional view of the outer skirt of FIG. 10.

FIG. 12 is an end view of the prosthetic heart valve of FIG. 10.

FIG. 13 illustrates the prosthetic heart valve of FIG. 10 positioned within an aortic annulus of a heart.

FIGS. 14A-14C illustrate a folding process for an outer skirt, according to one example.

FIG. 15 is an outer skirt for a prosthetic heart valve, according to one example.

FIG. 16 is a side view of an outer skirt coupled to a prosthetic heart valve, according to one example.

FIG. 17 is a partial cross-sectional view of the outer skirt of FIG. 16.

FIG. 18 is an end view of the prosthetic heart valve of FIG. 16.

FIG. 19 is a side view of an outer skirt coupled to a prosthetic heart valve, according to one example.

FIG. 20 is an end view of the prosthetic heart valve of FIG. 19.

FIG. 21 is a partial cross-sectional view of the outer skirt of FIG. 19.

FIG. 22 is a partial cross-sectional view of an outer skirt, according to one example.

FIG. 23 is a partial cross-sectional view of an outer skirt, according to one example.

FIG. 24 is a side view of an outer skirt coupled to a prosthetic heart valve, according to one example.

FIG. 25 is an end view of the prosthetic heart valve of FIG. 24.

FIG. 26 illustrates the prosthetic heart valve of FIG. 24 positioned within an aortic annulus of a heart.

FIG. 27 is a side view of an outer skirt coupled to a prosthetic heart valve according to one example.

FIG. 28 is an end view of the prosthetic heart valve of FIG. 27.

FIG. 29 is a side view of the prosthetic heart valve of FIG. 27 with the outer skirt stretched by a suture.

FIG. 30 is a side view of the prosthetic heart valve of FIG. 29 in a radially compressed state.

FIG. 31 is a side view of a delivery apparatus that includes the prosthetic heart valve of FIG. 30.

FIG. 32 is a side view of the prosthetic heart valve of FIG. 27 in a radially expanded state with the suture removed from the outer skirt.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

### Overview of the Disclosed Technology

Described herein are various examples of outer skirts (sealing members) for prosthetic valves that can be disposed around an outer surface of the frame of a prosthetic valve and can be configured to form a seal against native tissue upon implantation of the prosthetic valve. In this way, the outer skirts can reduce paravalvular leakage (PVL) past the prosthetic valve when expanded against the native anatomy. The outer skirts described herein can be configured to extend radially away from the frame when the prosthetic valve is radially expanded. As such, the outer skirts disclosed herein can form an improved seal against native tissue upon implantation of the prosthetic valve, and thus reduce PVL past the prosthetic valve.

Prosthetic valves disclosed herein can be radially compressible and expandable between a radially compressed state and a radially expanded state. Thus, the prosthetic valves can be crimped on or retained by an implant delivery apparatus in the radially compressed state while being advanced through a patient's vasculature on the delivery apparatus. In some examples, the outer skirts can be stretched in an axial direction when the prosthetic valve is in the radially compressed state to obtain a low-profile during delivery. For example, a suture can be attached to the outer skirt and extend proximally towards the implant delivery apparatus to stretch the outer skirt proximally over the frame. The prosthetic valve can be expanded to the radially expanded state once the prosthetic valve reaches the implantation site. The suture can also be removed from the outer skirt, thus allowing the skirt to retract distally and bulge radially outward to form a seal against the native tissue. It is understood that the prosthetic valves disclosed herein may be used with a variety of implant delivery apparatuses and can be implanted via various delivery procedures, examples of which will be discussed in more detail later.

### Examples of the Disclosed Technology

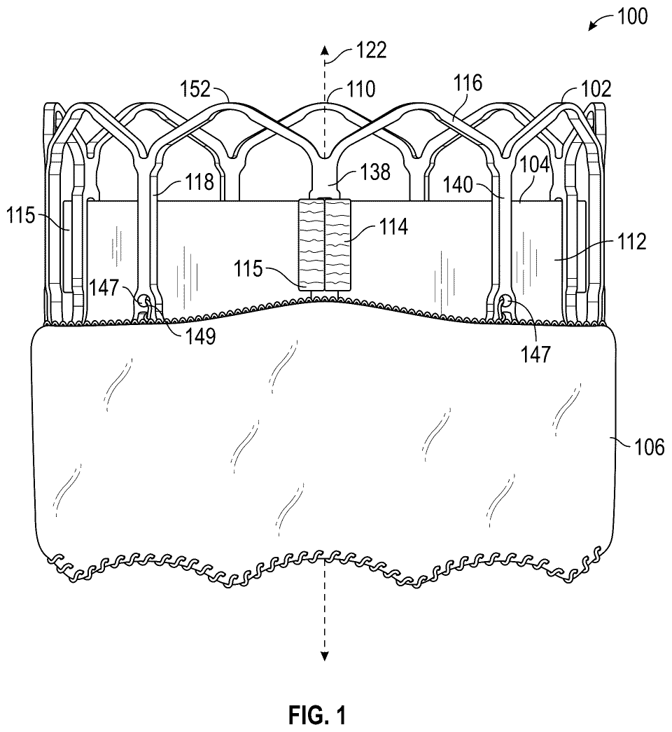

FIG. 1 shows a prosthetic heart valve 100 (prosthetic valve), according to one example. Any of the prosthetic valves disclosed herein are adapted to be implanted in the native aortic annulus, although in other examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In another example, the disclosed prosthetic valves can be implanted within a docking device implanted within or at the native mitral valve, such as disclosed in PCT Publication No. WO2020/247907, which is incorporated by reference herein. In another example, the disclosed prosthetic valves can be implanted within a docking device implanted within the superior or inferior vena cava for replacing the function of a diseased tricuspid valve, such as disclosed in U.S. Publication No. 2019/0000615, which is incorporated by reference herein.

The prosthetic heart valve 100 can include a stent or frame 102, a valvular structure 104, and a perivalvular outer sealing member or outer skirt 106. The prosthetic heart valve 100 (and the frame 102) can have an inflow end 108 and an outflow end 110. The valvular structure 104 can be disposed on an interior of the frame 102 while the outer skirt 106 is disposed around an outer surface of the frame 102.

The valvular structure 104 can comprise a plurality of leaflets 112 (e.g., three leaflets, as shown in FIG. 1), collectively forming a leaflet structure, which can be arranged to collapse in a tricuspid arrangement. The leaflets 112 can be secured to one another at their adjacent sides (e.g., commissure tabs) to form commissures 114 of the valvular structure 104. For example, each leaflet 112 can comprise opposing commissure tabs disposed on opposite sides of the leaflet 112 and a cusp edge portion extending between the opposing commissure tabs. The cusp edge portion of the leaflets 112 can have an undulating, curved scalloped shape, and can be secured directly to the frame 102 (e.g., by sutures). However, in alternate examples, the cusp edge portion of the leaflets 112 can be secured to an inner skirt which is then secured to the frame 102. In some examples, the leaflets 112 can be formed of pericardial tissue (e.g., bovine pericardial tissue), biocompatible synthetic materials, or various other suitable natural or synthetic materials as known in the art and described in U.S. Pat. No. 6,730,118, which is incorporated by reference herein.

In some examples, the outer skirt 106 can be an annular skirt. In some instances, the outer skirt 106 can comprise one or more skirt portions that are connected together and/or individually connected to the frame 102. The outer skirt 106 can comprise a fabric or a non-fabric material, which can be made of any of various polymers, such as ePTFE, PTFE, PET, TPU, UHMWPE, PEEK, PE, etc. In particular examples, the skirt 106 comprises PET fabric. In some examples, the outer skirt can be made of natural tissue, such as pericardium. In some instances, instead of having a relatively straight upper edge portion, as shown in FIG. 1, the outer skirt 106 can have an undulating upper edge portion that extends along and is secured to the angled struts 134. Examples of such outer skirts, as well as various other outer skirts, that can be used with the frame 102 can be found in U.S. Application No. 63/366,599, filed Jun. 17, 2022, which is incorporated by reference herein.

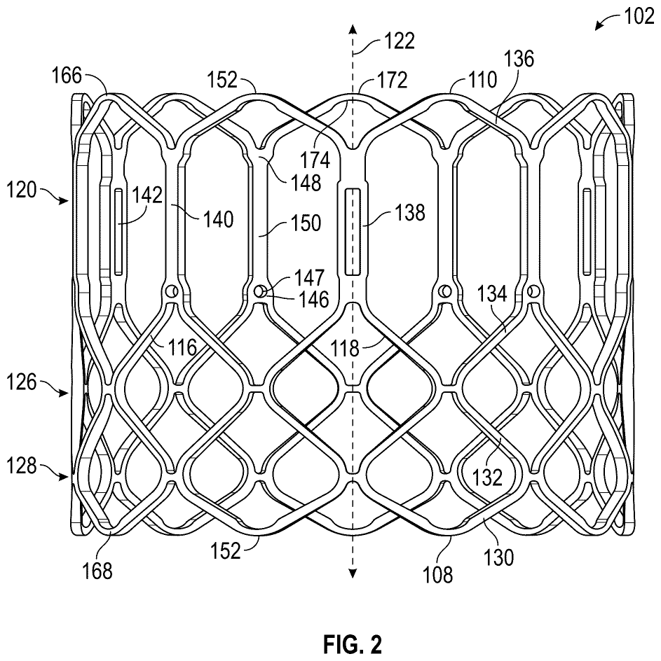

The frame 102 can be radially compressible and expandable between a radially compressed configuration and a radially expanded configuration (the expanded configuration is shown in FIG. 1). The frame 102 is shown alone in FIG. 2.

The frame 102 can be made of any of various suitable plastically-expandable materials (e.g., stainless steel, etc.) or self-expanding materials (e.g., nickel titanium alloy (NiTi), such as nitinol). When constructed of a plastically-expandable material, the frame 102 (and thus the valve 100) can be crimped to a radially compressed state on a delivery catheter and then expanded inside a patient by an inflatable balloon or equivalent expansion mechanism. When constructed of a self-expandable material, the frame 102 (and thus the valve 100) can be crimped to a radially compressed state and restrained in the compressed state by insertion into a sheath or equivalent mechanism of a delivery catheter. Once inside the body, the valve can be advanced from the delivery sheath, which allows the valve to expand to its functional size.

Suitable plastically-expandable materials that can be used to form the frames disclosed herein (for example, the frame 102) include, metal alloys, polymers, or combinations thereof. Example metal alloys can comprise one or more of the following: nickel, cobalt, chromium, molybdenum, titanium, or other biocompatible metal. In some examples, the frame 102 can comprise stainless steel. In some examples, the frame 102 can comprise cobalt-chromium. In some examples, the frame 102 can comprise nickel-cobalt-chromium. In some examples, the frame 102 comprises a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

As shown in FIG. 2, the frame 102 can comprise a plurality of interconnected struts 116 which form multiple rows of open cells 118 between the outflow end 110 and the inflow end 108 of the frame 102. In some examples, as shown in FIG. 2, the frame 102 can comprise three rows of cells 118 with a first (upper in the orientation shown in FIG. 2) row of cells 120 disposed at the outflow end 110. The first row of cells 120 comprises cells 118 that are elongated in an axial direction (relative to a central longitudinal axis 122 of the frame 102), as compared to cells 118 in the remaining rows of cells. For example, the cells 118 of the first row of cells 120 can have a longer axial length than cells 118 in the remaining rows of cells, which can include a second row of cells 126 and a third row of cells 128, the third row of cells 128 disposed at the inflow end 108 and the second row of cells 126 disposed between the first row of cells 120 and the third row of cells 128.

In some examples, as shown in FIG. 2, each row of cells comprises nine cells 118. Thus, in such examples, the frame 102 can be referred to as a nine-cell frame.

In alternate examples, the frame 102 can comprise more than three rows of cells (e.g., four or five) and/or more or less than nine cells per row. In some examples, the cells 118 in the first row of cells 120 may not be elongated compared to cells 118 in the remaining rows of cells of the frame 102 (the second row of cells 126 and the third row of cells 128).

The interconnected struts 116 can include a plurality of angled struts 130, 132, 134, and 136 arranged in a plurality of rows of circumferentially extending rows of angled struts, with the rows being arrayed along the length of the frame 102 between the outflow end 110 and the inflow end 108. For example, the frame 102 can comprise a first row of angled struts 130 arranged end-to-end and extending circumferentially at the inflow end 108 of the frame; a second row of circumferentially extending, angled struts 132; a third row of circumferentially extending, angled struts 134; and a fourth row of circumferentially extending, angled struts 136 at the outflow end 110 of the frame 102. The fourth row of angled struts 136 can be connected to the third row of angled struts 134 by a plurality of axially extending window struts 138 (or window strut portions) and a plurality of axial (e.g., axially extending) struts 140. The axially extending window struts 138 (which can also be referred to as axial struts that include a commissure window) define commissure windows (e.g., open windows) 142 that are spaced apart from one another around the frame 102, in a circumferential direction, and which are adapted to receive a pair of commissure tabs of a pair of adjacent leaflets 112 arranged into a commissure (e.g., commissure 114 shown in FIG. 1). In some examples, the commissure windows 142 and/or the axially extending window struts 138 defining the commissure windows 142 can be referred to herein as commissure features or commissure supports, each commissure feature or support configured to receive and/or be secured to a pair of commissure tabs of a pair of adjacent leaflets.

One or more (e.g., two, as shown in FIG. 2) axial struts 140 can be positioned between, in the circumferential direction, two commissure windows 142 formed by the window struts 138. Since the frame 102 can include fewer cells per row (e.g., nine) and fewer axial struts 140 between each commissure window 142, as compared to some more traditional prosthetic heart valves, each cell 118 can have an increased width (in the circumferential direction), thereby providing a larger opening for blood flow and/or coronary access.

Each axial strut 140 and each window strut 138 extends from a location defined by the convergence of the lower ends (e.g., ends arranged inward of and farthest away from the outflow end 110) of two angled struts 136 (which can also be referred to as an upper strut junction or upper elongated strut junction) to another location defined by the convergence of the upper ends (e.g., ends arranged closer to the outflow end 110) of two angled struts 134 (which can also be referred to as a lower strut junction or lower elongate strut junction). Each axial strut 140 and each window strut 138 forms an axial side of two adjacent cells of the first row of cells 120.

Commissure tabs 115 of adjacent leaflets 112 can be secured together to form commissures 114 (FIG. 1). Each commissure 114 of the prosthetic heart valve 100 comprises two commissure tabs 115 paired together, one from each of two adjacent leaflets 112, and extending through a commissure window 142 of the frame 102. Each commissure 114 can be secured to the window struts 138 forming the commissure window 142.

The cusp edge portion (e.g., scallop edge) of each leaflet 112 can be secured to the frame 102 via one or more fasteners (e.g., sutures). In some examples, the cusp edge portion of each leaflet 112 can be secured directly to the struts of the frame 102 (e.g., angled struts 130, 132, and 134). For example, the cusp edge portions of the leaflets 112 can be sutured to the angled struts 130, 132, and 134 that generally follow the contour of the cusp edge portions of the leaflets 112.

In some examples, the cusp edge portion of the leaflets 112 can be secured to an inner skirt and the inner skirt can then be secured directly to the frame 102.

Various methods for securing the leaflets 112 to a frame, such as the frame 102, are disclosed in U.S. provisional patent applications 63/278,922, filed Nov. 12, 2021, and 63/300,302, filed Jan. 18, 2022, both of which are incorporated by reference herein.

As shown in FIG. 2, in some examples, one or more of or each of the axial struts 140 can comprise an inflow end portion 146 (e.g., an end portion that is closest to the inflow end 108) and an outflow end portion 148 that are widened relative to a middle portion 150 of the axial strut 140. In some instances, the inflow end portion 146 of the axial strut 140 can comprise an aperture 147. The apertures 147 can be configured to receive fasteners (e.g., sutures) for attaching soft components of the prosthetic heart valve 100 to the frame 102. For example, in some instances, the outer skirt 106 can be positioned around the outer surface of the frame 102 and an upper or outflow edge portion of the outer skirt 106 can be secured to the apertures 147 by fasteners 149 (e.g., sutures), as shown in FIG. 1.

The frame 102 can further comprise a plurality of apex regions 152 formed at the inflow end 108 and the outflow end 110, each apex region 152 extending and forming a junction between two angled struts 130 at the inflow end 108 or two angled struts 136 at the outflow end 110. As such, the apex regions 152 are spaced apart from one another, in a circumferential direction at the inflow end 108 and the outflow end 110.

Each apex region 152 and two corresponding angled struts 136 at the outflow end 110 can form an outflow strut 166 and each apex region 152 and two corresponding angled struts 130 at the inflow end 108 can form an inflow strut 168.

Each outflow strut 166 and inflow strut 168 can have a length that includes an apex region 152 and the two angled struts 136 or 130 (or strut portions), respectively, on either side of the apex region 152.

In some examples, each apex region 152 can comprise a curved, axially facing outer surface 172 and an arcuate or curved, axially facing inner depression 174 which forms thinned strut portions of the apex regions 152. For example, the curved inner depression 174 can depress toward the curved outer surface 172 from an inner surface of the angled strut portions 156, thereby forming the smaller width thinned strut portions. Thus, the curved inner depressions 174 can be formed on a cell side of the apex region 152 (e.g., as opposed to the outside of the apex region 152).

In some examples, the curved outer surface 172 of each apex region 152 can form a single, continuous curve from one angled strut portion 156 on a first side of the apex region 152 to another angled strut portion 156 on an opposite, second side of the apex region 152.

Additional details and examples of frames for prosthetic heart valves that include apex regions can be found in U.S. Provisional Patent Application Nos. 63/178,416, filed Apr. 22, 2021, 63/194,930, filed May 28, 2021, and 63/279,096, filed Nov. 13, 2021, all of which are incorporated by reference herein.

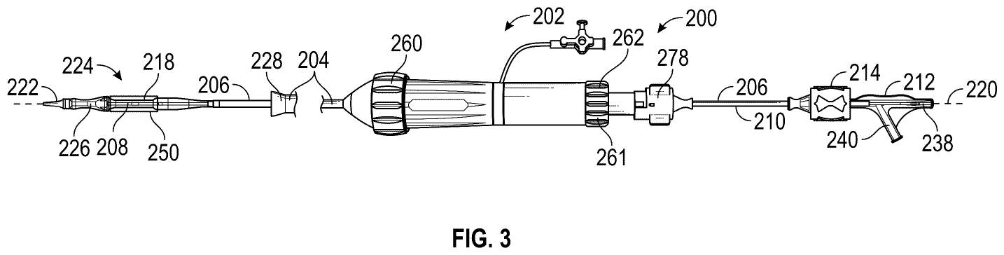

FIG. 3 shows a delivery apparatus 200, according to an example, that can be used to implant an expandable prosthetic heart valve (e.g., the prosthetic heart valve 100 of FIG. 1 and/or any of the other prosthetic heart valves described herein). In some examples, the delivery apparatus 200 is specifically adapted for use in introducing a prosthetic valve into a heart.

The delivery apparatus 200 in the illustrated example of FIG. 3 is a balloon catheter comprising a handle 202 and a steerable, outer shaft 204 extending distally from the handle 202. The delivery apparatus 200 can further comprise an intermediate shaft 206 (which also may be referred to as a balloon shaft) that extends proximally from the handle 202 and distally from the handle 202, the portion extending distally from the handle 202 also extending coaxially through the outer shaft 204. Additionally, the delivery apparatus 200 can further comprise an inner shaft 208 extending distally from the handle 202 coaxially through the intermediate shaft 206 and the outer shaft 204 and proximally from the handle 202 coaxially through the intermediate shaft 206.

The outer shaft 204 and the intermediate shaft 206 can be configured to translate (e.g., move) longitudinally, along a central longitudinal axis 220 of the delivery apparatus 200, relative to one another to facilitate delivery and positioning of a prosthetic valve at an implantation site in a patient's body.

The intermediate shaft 206 can include a proximal end portion 210 that extends proximally from a proximal end of the handle 202, to an adaptor 212. A rotatable knob 214 can be mounted on the proximal end portion 210 and can be configured to rotate the intermediate shaft 206 around the central longitudinal axis 220 and relative to the outer shaft 204.

The adaptor 212 can include a first port 238 configured to receive a guidewire therethrough and a second port 240 configured to receive fluid (e.g., inflation fluid) from a fluid source. The second port 240 can be fluidly coupled to an inner lumen of the intermediate shaft 206.

The intermediate shaft 206 can further include a distal end portion that extends distally beyond a distal end of the outer shaft 204 when a distal end of the outer shaft 204 is positioned away from an inflatable balloon 218 of the delivery apparatus 200. A distal end portion of the inner shaft 208 can extend distally beyond the distal end portion of the intermediate shaft 206.

The balloon 218 can be coupled to the distal end portion of the intermediate shaft 206.

In some examples, a distal end of the balloon 218 can be coupled to a distal end of the delivery apparatus 200, such as to a nose cone 222 (as shown in FIG. 3), or to an alternate component at the distal end of the delivery apparatus 200 (e.g., a distal shoulder). An intermediate portion of the balloon 218 can overlay a valve mounting portion 224 of a distal end portion of the delivery apparatus 200 and a distal end portion of the balloon 218 can overly a distal shoulder 226 of the delivery apparatus 200. The valve mounting portion 224 and the intermediate portion of the balloon 218 can be configured to receive a prosthetic heart valve in a radially compressed state. For example, as shown schematically in FIG. 3, a prosthetic heart valve 250 (which can be one of the prosthetic valves described herein) can be mounted around the balloon 218, at the valve mounting portion 224 of the delivery apparatus 200.

The balloon shoulder assembly, including the distal shoulder 226, is configured to maintain the prosthetic heart valve 250 (or other medical device) at a fixed position on the balloon 218 during delivery through the patient's vasculature.

The outer shaft 204 can include a distal tip portion 228 mounted on its distal end. The outer shaft 204 and the intermediate shaft 206 can be translated axially relative to one another to position the distal tip portion 228 adjacent to a proximal end of the valve mounting portion 224, when the prosthetic valve 250 is mounted in the radially compressed state on the valve mounting portion 224 (as shown in FIG. 3) and during delivery of the prosthetic valve to the target implantation site. As such, the distal tip portion 228 can be configured to resist movement of the prosthetic valve 250 relative to the balloon 218 proximally, in the axial direction, relative to the balloon 218, when the distal tip portion 228 is arranged adjacent to a proximal side of the valve mounting portion 224.

An annular space can be defined between an outer surface of the inner shaft 208 and an inner surface of the intermediate shaft 206 and can be configured to receive fluid from a fluid source via the second port 240 of the adaptor 212. The annular space can be fluidly coupled to a fluid passageway formed between the outer surface of the distal end portion of the inner shaft 208 and an inner surface of the balloon 218. As such, fluid from the fluid source can flow to the fluid passageway from the annular space to inflate the balloon 218 and radially expand and deploy the prosthetic valve 250.

An inner lumen of the inner shaft can be configured to receive a guidewire therethrough, for navigating the distal end portion of the delivery apparatus 200 to the target implantation site.

The handle 202 can include a steering mechanism configured to adjust the curvature of the distal end portion of the delivery apparatus 200. In the illustrated example, for example, the handle 202 includes an adjustment member, such as the illustrated rotatable knob 260, which in turn is operatively coupled to the proximal end portion of a pull wire. The pull wire can extend distally from the handle 202 through the outer shaft 204 and has a distal end portion affixed to the outer shaft 204 at or near the distal end of the outer shaft 204. Rotating the knob 260 can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein.

The handle 202 can further include an adjustment mechanism 261 including an adjustment member, such as the illustrated rotatable knob 262, and an associated locking mechanism including another adjustment member, configured as a rotatable knob 278. The adjustment mechanism 261 is configured to adjust the axial position of the intermediate shaft 206 relative to the outer shaft 204 (e.g., for fine positioning at the implantation site). Further details on the delivery apparatus 200 can be found in PCT Application No. PCT/US2021/047056, which is incorporated by reference herein.

As introduced above, when a prosthetic valve (for example, prosthetic valve 100, etc.) is expanded within a native heart valve, an outer skirt (for example, outer skirt 106, etc.) of the prosthetic valve can contact tissue of the surrounding native valve to establish a seal between the prosthetic valve and the surrounding tissue that prevents or reduces paravalvular leakage. Depending on the patient's anatomy, the native valve can have an irregular shape, such as due to the presence of calcium nodules, which can prevent the outer skirt from fully sealing against the surrounding tissue. As such, it is desirable for an outer skirt to conform and seal around any native valve, including an irregularly shaped or calcified native valve.

In some examples, an outer skirt for a prosthetic heart valve (e.g., prosthetic heart valve 100) can include one or more pleats, folds, or loops of material that extend radially away from a frame of the prosthetic heart valve. The pleats of the outer skirt be configured to compress against tissue of a native annulus (e.g., by different amounts dependent on the adjacent tissue, etc.) to conform to any irregularities present within the native annulus. As a result, an improved seal between the outer skirt and the native annulus can be achieved, thereby reducing paravalvular leakage.

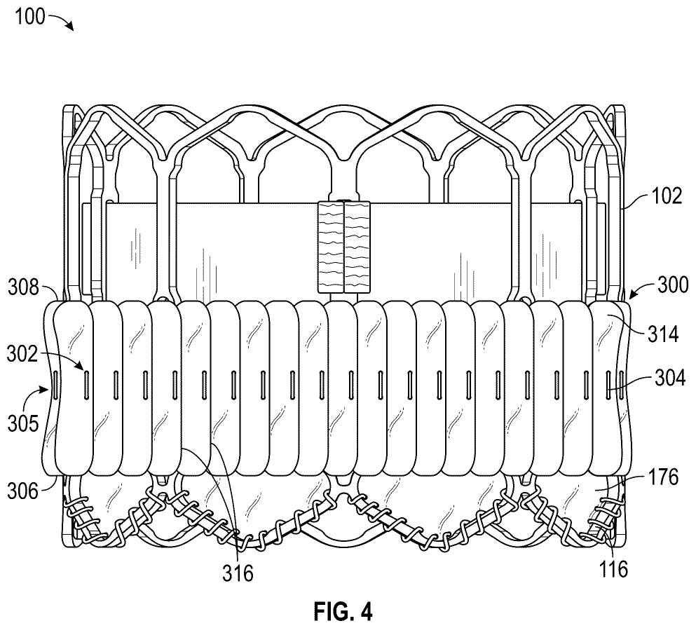

FIGS. 4-7 show one example of an outer skirt, or sealing member, 300 coupled to the prosthetic heart valve 100 of FIG. 1 (for example, in lieu of outer skirt 106). As described in further detail below, the outer skirt 300 can comprise a plurality of pleats extending radially outward from an outer surface of the frame of the prosthetic valve. While shown as coupled to prosthetic heart valve 100, the outer skirt 300 can be used in a mechanically expandable prosthetic valve, other balloon-expandable prosthetic valves (e.g., other than prosthetic heart valve 100 of FIG. 1), and/or a self-expandable prosthetic valve. Additional details on balloon expandable prosthetic valves can be found in U.S. Pat. No. 9,393,110, and U.S. Provisional Application Nos. 63/178,416, filed Apr. 22, 2021, 63/194,930, filed May 28, 2021, and 63/279,096, filed Nov. 13, 2021, as already incorporated by reference herein. Additional details on a mechanically expandable prosthetic valve can be found in International Application PCT/US2021/052745, filed Sep. 30, 2021, which is incorporated by reference herein. Additional details on a self-expanding prosthetic valve can be found in U.S. Pat. No. 8,652,202, which is incorporated by reference herein.

The prosthetic heart valve 100 can include an inner skirt 176 coupled to an inner surface of the frame 102. In some examples, as shown in FIG. 4, the inner skirt 176 can coupled to the struts 116 at the inflow end 108 of the prosthetic heart valve 100.

The outer skirt 300 is disposed around an outer surface of the frame 102 and can be coupled to the frame 102 with one or more sutures 302 forming a plurality of stitches 304. For example, the stitches 304 can extend through a frame attachment region 305 of the skirt 300 and around one or more struts 116 of the frame 102. The stitches 304 can be spaced apart from each other in a circumferential direction around the skirt 300. The frame attachment region 305 can be disposed between an inflow edge 306 of the skirt 300 and an outflow edge 308 of the skirt 300. In some examples, as depicted, a single row of stitches 304 extends circumferentially at an axial mid-point of the skirt 300, thereby dividing the skirt 300 into two equal portions. In other examples, the frame attachment region 305 (and one or more rows of stitches 304) can be positioned at other axial positions between the inflow edge 306 and the outflow edge 308, for example, to divide the skirt 300 into multiple equal or unequal portions. In other examples, the frame attachment region 305 (and the stitches 304) can be positioned at the inflow edge 306 and/or the outflow edge 308 of the skirt 300.

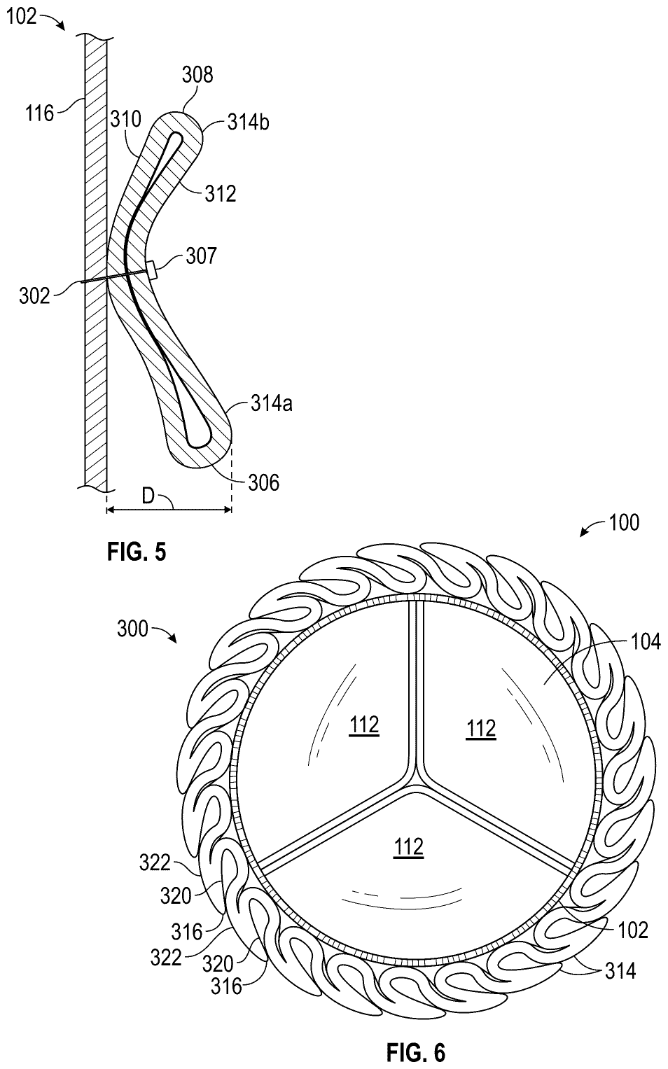

The skirt 300 can comprise a single layer of material or a plurality of layers. The skirt 300 can be made from any of the materials disclosed above for skirt 106. For example, as shown in FIG. 5, the skirt 300 can comprise an inner layer 310 and an outer layer 312 positioned radially outside of the inner layer 310. As described in detail below, the skirt 300 can be formed from a single piece of material that is folded to form the inner and outer layers 310, 312. In other examples, the inner and outer layers 310, 312 can be separate pieces of material. In the illustrated example, the inner layer 310 is adjacent to the outer layer 312. In other examples, one or more additional layers can be positioned between the inner layer 310 and the outer layer 312. For example, additional layer(s) of the skirt 300 can be disposed between the layers 310, 312 and/or one or more spacers can be positioned between the layers 310, 312, as described in more detail below. In some examples, additional layer(s) can be disposed radially inward of the inner layer 310 and/or radially outward of the outer layer 312.

The outer skirt 300 can comprise a plurality of longitudinally extending folds or pleats 314. The pleats 314 are formed along longitudinally extending fold lines, and therefore can be referred to as longitudinal pleats or folds. In the illustrated example, when the skirt 300 is coupled to the frame 102 by the one or more sutures 302, each pleat 314 defines two pleat portions that include at least part of the inner layer 310 and at least part of the outer layer 312. Specifically, as shown in FIG. 5, the skirt 300 comprises a first (or inflow) pleat portion 314a extending axially from the frame attachment region 305 towards the inflow edge 306 and a second (or outflow) pleat portion 314b extending axially from frame attachment region 305 towards the outflow edge 308. The skirt 300 can define any number of pleats 314, for example, based on the location of the frame attachment region 305, the placement of the stitches 304 and/or number of stitches 304, etc.

In some examples, a plurality of sutures 302 form discrete stitches 304 that are spaced apart from each other in a circumferential direction of the skirt 300. As shown in FIG. 5, each stitch 304 can extend through the inner and outer layers 310, 312 and around a portion of the frame 102 (for example, an adjacent strut 116), and the ends of the stitch can be tied off to each other to form a knot 307. In other examples, a single suture 302 can be used for a plurality of continuous stitches 304 that extend through the skirt and around portions of the frame along a circumferentially extending stitch line.

The skirt 300 can be configured to protrude radially outward from the frame 102. In some instances, as shown, the skirt 300 can have one frame attachment region 305 secured to the frame 102 with stitches 304, which can be the only location where the pleats 314 are coupled to the frame 102. In this way, the inflow and outflow edges 306, 308 of the pleats 314 are free to protrude radially outward from the frame 102. For example, the pleats 314 can be configured to protrude radially outward from the outer surface of the frame 102 by a distance D (FIG. 5). In some examples, the distance D can be, for example, in the range of about 0.5 mm to about 2 mm.

As best shown in FIG. 6, each pleat 314 can be wrapped or folded in a circumferential direction such that each pleat 314 overlaps an adjacent pleat 314 in a circumferential direction and forms an inner pleat section 320 and an outer pleat section 322 positioned radially outward of the inner pleat section 320. The inner pleat section 320 and the outer pleat section 322 are joined to each other at an axially extending edge 316 formed at a fold line of the pleat. In other examples, the pleats 314 extend radially outwardly from the frame without any overlap between adjacent pleats when the prosthetic valve is radially expanded.

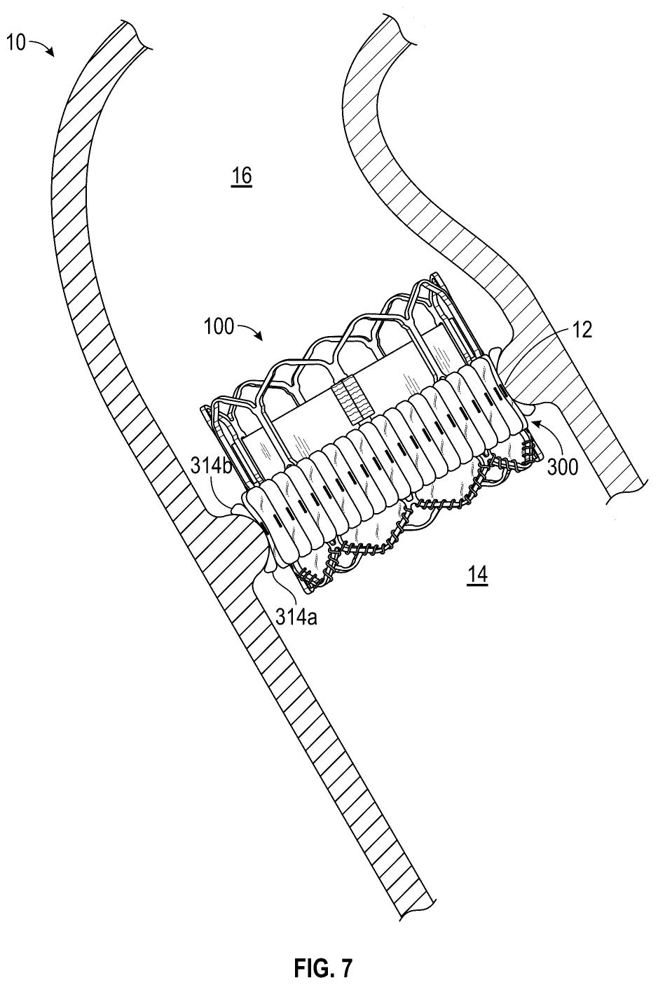

FIG. 7 illustrates the prosthetic valve 100 having skirt 300 as implanted within a heart 10 of a patient. As shown, the skirt 300 can be positioned adjacent to the native aortic annulus 12 (shown schematically without the native leaflets for purposes of illustration), such that the pleat portions 314a, 314b can protrude radially away from the frame 102 and at least partially against the annulus 12. For example, the inflow pleat portion 314a can form a seal with the inflow portion of the annulus 12 and can extend slightly into the left ventricle 14 and the outflow pleat portion 314b can form a seal with the outflow portion of annulus 12 and can extend slightly into the aorta 16.

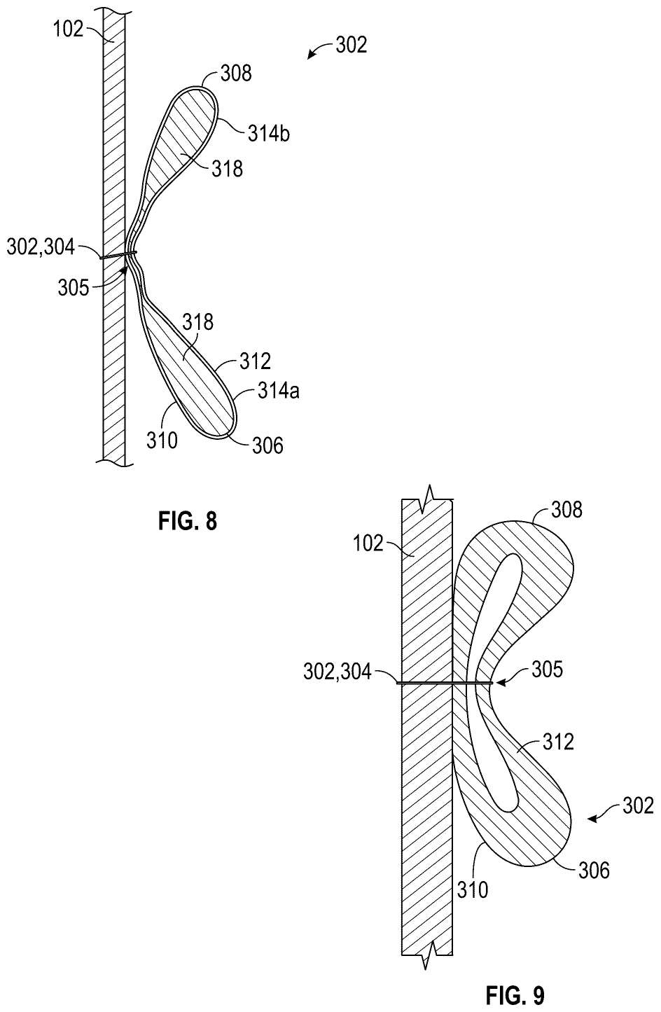

In some examples, as depicted in FIG. 8, the skirt 300 can optionally include a spacer 318 positioned within an internal space of the skirt 300. For example, the spacer 318 can be disposed between the inner layer 310 and the outer layer 312. The spacer 318 can comprise a compressible material, for example, a compressible silicone, foam (for example, a polyurethane foam), gel material, such as a hydrogel, and/or one or more pieces of fabric or natural tissue. In this way, the skirt 300 can be expanded radially outwardly by the spacer 318 and can also be selectively compressed by a patient's native anatomy at the implantation site, to help improve the seal between the skirt 300 and the implantation site.

A spacer 318 can be disposed within each of the pleat portions 314a, 314b. For example, as depicted in FIG. 8, the skirt 300 can include two spacers 318, with one positioned axially towards the inflow edge 306 relative to the frame attachment region 305 and the other positioned axially towards the outflow edge 308 relative to the frame attachment region 305. In some examples, the skirt 300 does not include a spacer 318 disposed within the internal space of the skirt 300 where the stitches 304 pass through the inner and outer layers 310, 312. This can enable the skirt 300 to be smaller in a radial direction at the frame attachment region 305 (the portion of the skirt 300 where the stitches 304 couple the skirt 300 to the frame 102) than towards the inflow and outflow edges 306, 308 of the skirt. In some examples, the spacer 318 can be positioned within the skirt 300 at the frame attachment region 305, for example, such that the spacer 318 extends from the inflow edge 306 to the outflow edge 308. In such examples, the radial size of the skirt 300 can be generally uniform in the axial direction.

In some examples, the skirt 300 can include layers 310, 312 that have a varied thickness. For example, as shown in FIG. 9, the skirt 300 can be radially thicker at the inflow and outflow edges 306, 308 and can be radially thinner at the frame attachment region 305. In this way, the portions of the skirt 300 that are free relative to the frame 102 (e.g., the inflow and outflow edges 306, 308) can extend radially away from the frame 102 by a relatively increased distance based on the thickness of the material, while allowing a thinner frame attachment region 305 for the stitches 304 to pass through. In some instances, the thickness of the material at a given location can correspond to a number of layers of the skirt 300 at that location. For example, one layer or multiple layers coupled together can be used to achieve the desired thickness.

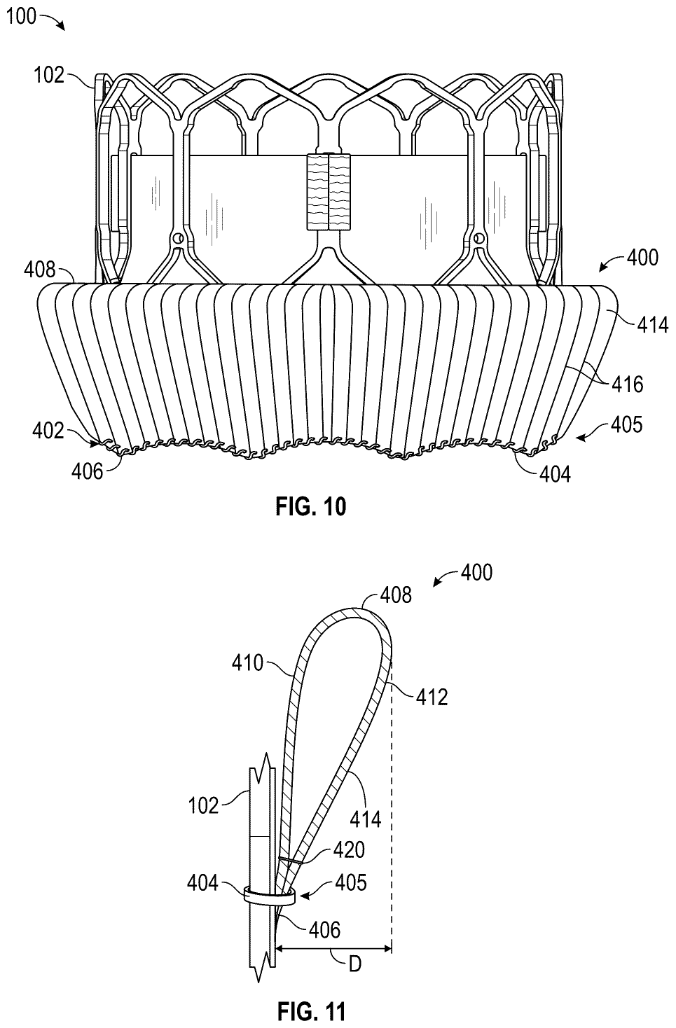

FIGS. 10-13 show an example of an outer skirt, or sealing member, 400 coupled to the prosthetic heart valve 100 of FIG. 1 (for example, in lieu of outer skirt 106). The skirt 400 can be made from any of the materials disclosed above for skirt 106. The outer skirt 400 is similar to outer skirt 300, except that each pleat includes one pleat portion extending radially outward from an outer surface of the frame 102, rather than two or more. While shown as coupled to prosthetic heart valve 100, the outer skirt 400 can be used in a mechanically expandable prosthetic valve, other balloon-expandable prosthetic valves (e.g., other than prosthetic heart valve 100 of FIG. 1), and/or a self-expandable prosthetic valve.

Similar to skirt 300, the outer skirt 400 can be coupled to the outer surface of the frame 102 with one or more sutures 402 forming a plurality of stitches 404. For example, the stitches 404 can extend through a frame attachment region 405 of the skirt 400 and around adjacent portions the frame 102 (for examples, struts 116). The stitches 404 can form a stitch line that extends in a circumferential direction around the skirt 400. In the illustrated example, the stitch line comprises a plurality of continuous stitches 404 formed around a circumference of the skirt. In other examples, the stitches 404 can be discrete stitches that are spaced apart from each other in the circumferential direction. In some examples, as depicted, the frame attachment region 405 can be at an inflow edge 406 of the skirt 400. Alternatively, the frame attachment region 405 can be at an outflow edge 408 of the skirt 400.

The skirt 400 can comprise a single layer or a plurality of layers. For example, as shown in FIG. 11, the skirt 400 can comprise an inner layer 410 and an outer layer 412 positioned radially outside of the inner layer 410. In the illustrated example, the inner layer 410 is adjacent to the outer layer 412. In other examples, one or more additional layers can be positioned between the inner layer 410 and the outer layer 412. For example, additional layer(s) of the skirt 400 can be disposed between the layers 410, 412 and/or one or more spacers (such as spacers 318) can be positioned between the layers 410, 412, as described above. In some examples, additional layer(s) can be disposed radially inward of the inner layer 410 and/or radially outward of the outer layer 412.

As shown, the outer skirt 400 can comprise a plurality of longitudinal pleats 414. Each pleat 414 can comprise at least part of the inner layer 410 and at least part of the outer layer 412 and can extend axially from the frame attachment region 405 towards outflow edge 408.

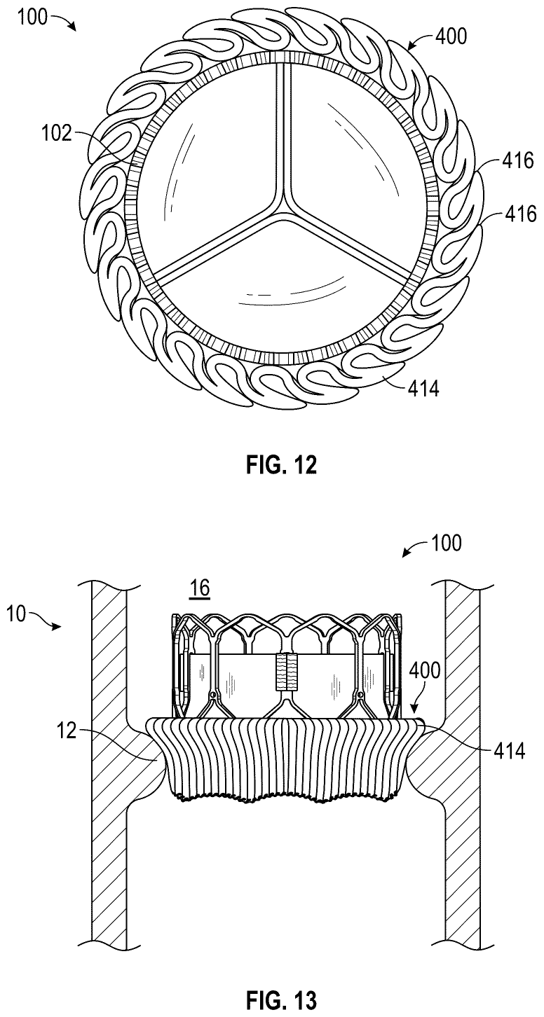

The skirt 400 can be configured to protrude radially outward from the frame 102. For example, as shown in FIGS. 11-12, the pleats 414 can be configured to protrude radially outward from the outer surface of the frame 102 by a distance D. In some instances, as shown, the skirt 400 can have one frame attachment region 405 at the inflow edge 406 and the stitches 404 can be the only location where the pleats 414 are coupled to the frame 102. In this way, the outflow edge 408 of the pleat 414 is free to protrude outward from the frame 102.

Similar to skirt 300, the skirt 400 can comprise a plurality of axially extending edges 416, and the pleats 414 can be wrapped or folded circumferentially such that each pleat 414 at least partially overlaps an adjacent pleat 414 in the circumferential direction.

FIG. 13 illustrates the prosthetic valve 100 having skirt 400 as implanted within a heart 10 of a patient. As shown, the skirt 400 can be positioned adjacent to the native aortic annulus 12 (shown schematically without the native leaflets for purposes of illustration), such that the pleats 414 can protrude radially away from the frame 102 and at least partially against the annulus 12. For example, the pleats 414 can form a seal with the annulus 12 along the supra annulus surface of the annulus 12 within the aorta 16.

The skirt 400 can comprise an internal space between the inner layer 410 and the outer layer 412. In some examples, not shown, the skirt 400 can optionally include a spacer (such as spacer 318) positioned within the internal space to help improve the seal of the skirt 400.

The skirt 400 can be formed by folding a piece of material into a flattened tubular shape. Circumferential (or lengthwise) edges of the layers 410, 412 can be folded towards each other, for example, towards the frame attachment region 405 of the skirt 400. The layers 410, 412 can be coupled together and/or with one edge coupled to the opposite layer (either layer 410 or 412) (for example, such that the edges are axially offset). In some examples, the inner and outer layers 410, 412 can be coupled together with stitches 420 adjacent to the frame attachment region 405, for example, positioned on a side of the region 405 towards the outflow edge 408. In some instances, as depicted, the stitches 420 used to couple the inner and outer layers 410, 412 together are separate from the stitches 404 that couple the skirt 400 to the frame 102. In some instances, the same stitches (e.g., stitches 404) can couple the inner and outer layers 410, 412 together as well as to the frame 102.

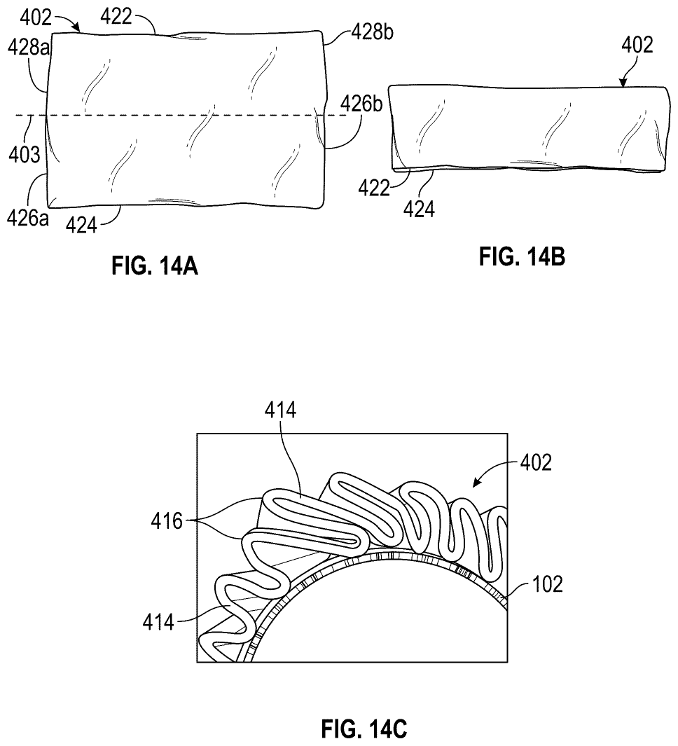

FIGS. 14A-14C illustrate an exemplary method of folding a piece of material 403 (for example, a piece of fabric) to create an outer skirt 400 with overlapping pleats 414. Initially, the material 403 is folded along a fold line 407 to align a first edge 422 (upper edge in FIG. 14A) with a second edge 424 (lower edge in FIG. 14A), such that the material 403 is folded in half or approximately in half (FIG. 14B). Folding the edges 422, 424 together creates a flattened tubular shape or a dual layer sheet comprising inner and outer layers corresponding the inner layer 410 and the outer layer 412 of the fully formed skirt 400. In some examples, the edges 422, 424 can be offset from each other in the axial direction when folded, such that the skirt comprises only one layer at a frame attachment region.

As described above, the inner and outer layers can be secured together, such as with sutures, along the periphery of the two layers. For example, the first and second edges 422, 424 can be stitched together along the length of the edges 422, 424. Further, side edge 426a of the inner layer can be stitched to side edge 428a of the outer layer along the length of the edges 426a, 428a, and side edge 426b of the inner layer can be stitched to side edge 428b of the outer layer. In some examples, to form an annular shape, side edge 426a of the inner layer can be stitched to side edge 426b of the inner layer along the length of the edges 426a, 426b, and side edge 428a of the outer layer can be stitched to side edge 428b of the outer layer. In other examples, one or more of the opposing edges can be left unsecured to each other. In some examples, another layer or material (e.g., spacer 318) can be positioned between the inner and outer layers prior to securing the inner and outer layers together and/or to a frame.

Either before or after the inner and outer layers are coupled together with sutures, the folded dual-layer piece of material 403 can be folded along axial fold lines to form multiple overlapping pleats (corresponding to pleats 414) and placed around a frame 102, as shown in FIG. 14C. The axial fold lines can form the axial edges 416 of the pleats 414 of the fully formed skirt 400. As shown in FIG. 14C, in some examples, the pleats 414 and/or the edges 416 can be non-uniformly spaced apart in the circumferential direction, such that the size of the pleats 414 is varied. In some examples (FIG. 12), the pleats 414 and the edges 416 can be uniformly spaced apart in the circumferential direction around the frame 102, such that the pleats 414 are a uniform size. After the overlapping pleats 414 are formed, the pleated skirt 400 can be coupled to the frame 102 with stitches 404. In some examples, the stitches 404 can couple the inner and outer layers together. While skirt 400 is shown, a similar folding pattern can be used to create outer skirt 300 with overlapping pleats 314.

In some examples, as depicted in FIGS. 14A-14C, a single piece of material 403 is folded into an annular shape with overlapping pleats 414, for example, with the edges 426a, 426b, 428a, 428b of the material 403 coupled together. In some examples, two or more pieces of material 403 can be coupled together at adjacent side edges to be the desired length (based on a desired circumference of the fully formed skirt) and subsequently folded to form the overlapping pleats.

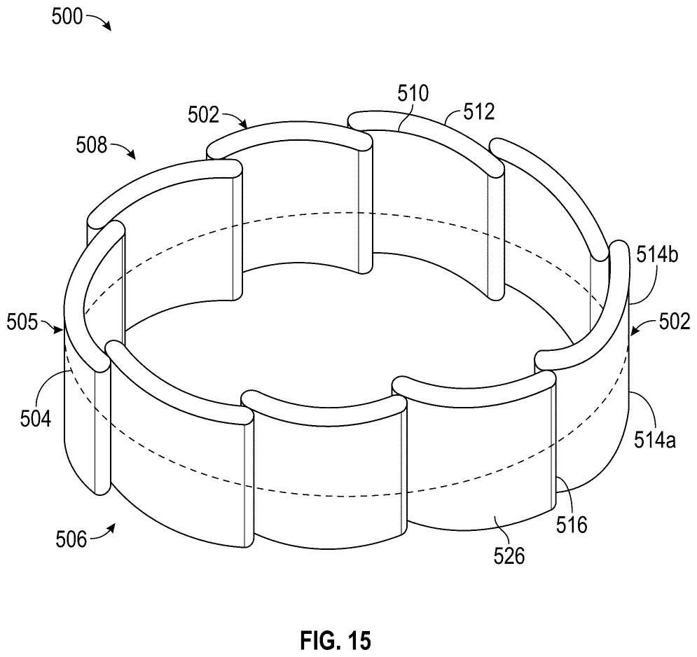

In some instances, longitudinal edges of an outer skirt (such as outer skirts 300, 400) can be defined by multiple, discrete segments of material that are overlapping. For example, as depicted in FIG. 15, an outer skirt, or sealing member, 500 can comprise a plurality of tubular shape segments 526 (or “tube segment”), each of which can be partially layered in the radial direction over at least one other segment 526. Each segment 526 can have an inner layer 510 and an outer layer 512 that is positioned radially outwards of the inner layer 510. For example, each segment 526 can comprise a material that is folded or pleated about an axial fold line to form the inner layer 510 and the outer layer 512, such that the inner layer 510 and the outer layer 512 are joined to each other at an axially extending edge 516. While outer skirt 500 is shown alone, it should be appreciated that outer skirt 500 can be coupled to a frame of a prosthetic valve as previously described, similar to outer skirts 300 and 400. The skirt 500 can be made from any of the materials disclosed above for skirt 106. It should be appreciated that longitudinal edges 316, 416 of outer skirts 300, 400 can be defined by overlapping tube segments, similar to segments 526, rather than the folded pleats described above.

The outer skirt 500 can be coupled to a frame with one or more sutures 502 forming a plurality of stitches 504. The stitches 504 can extend through a frame attachment region 505 of the skirt 500. When the frame attachment region 505 is positioned at a mid-section of the skirt 500, as depicted, each segment 526 can define an inflow pleat 514a adjacent to the inflow edge 506 and outflow pleat 514b adjacent to the outflow edge 508. In other examples, the frame attachment region 505 can be positioned at the inflow edge 506 (similar to outer skirt 400) or the outflow edge 508.

Each segment 526 can be open at the inflow edge 506 and at the outflow edge 508, such that the inner layer 510 is radially separate from the outer layer 512 at the edges 506, 508. In other examples, each segment 526 can be closed, with the inner and outer layers 510, 512 coupled together at the edges 506, 508, for example, to retain an optional spacer within an internal space defined between the inner and outer layers 510, 512 of the skirt 500. While the segments 526 are shown as oriented with folded edges 516 extending in an axial direction, in other instances, the segments 526 can be oriented in other directions (for example, with folded edges 516 of the segments 526 at the inflow and outflow edges 506, 508 of the skirt 500).

In some examples, each segment 526 can be folded to create overlapping pleats (similar to pleats 414) such that each segment 526 overlaps a portion of itself as well as an adjacent segment 526. For example, one or more segments can include longitudinal folds, such that at least some of the tubular segments of an outer skirt are both folded and overlapping.

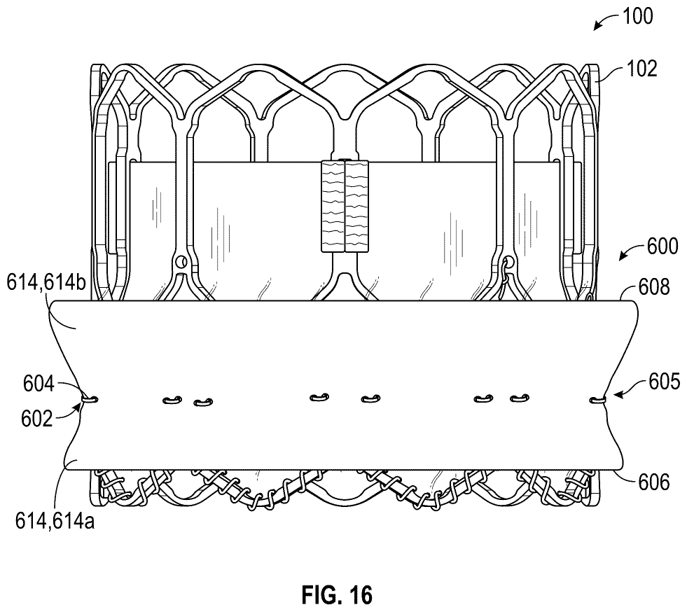

FIGS. 16-18 illustrate another example of an outer skirt, or sealing member, 600 for a prosthetic heart valve. The outer skirt 600 is similar to skirt 300 but does not include longitudinal edges 316, for example. Rather than having overlapping pleats and/or discrete tube segments to define longitudinal edges, the outer skirt 600 is smooth and continuous in the circumferential direction. While shown as coupled to prosthetic heart valve 100, the outer skirt 600 can be used in a mechanically expandable prosthetic valve, other balloon-expandable prosthetic valves (e.g., other than prosthetic heart valve 100 of FIG. 1), and/or a self-expandable prosthetic valve.

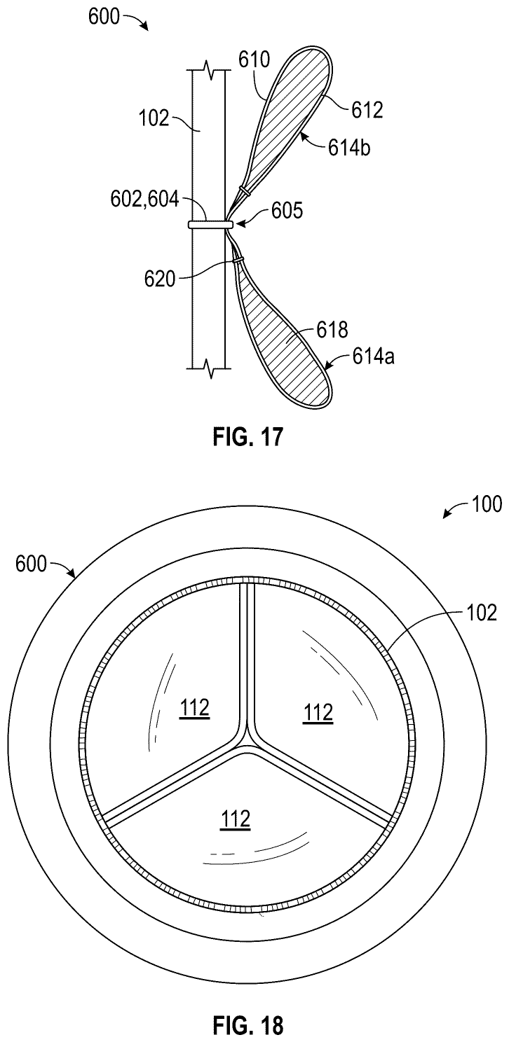

As introduced above, the outer skirt 600 can be disposed around an outer surface of the frame 102. The skirt 600 can be coupled to the frame 102 with one or more sutures 602 forming a plurality of stitches 604. For example, the stitches 604 can extend through a frame attachment region 605 of the skirt 600 and around one or more struts 116 of the frame 102. The stitches 604 can extend in a circumferential direction around the skirt 600. As shown in FIG. 16, the frame attachment region 605 is disposed between an inflow edge 606 of the skirt 600 and an outflow edge 608 of the skirt 600. In some examples, as depicted, a single row of stitches 604 extends circumferentially at an axial mid-point of the skirt 600, thereby dividing the skirt 600 into two equal (or approximately equal) portions. The skirt 600 can comprise a plurality of layers including, for example, a radially inner layer 610 and a radially outer layer 612, as shown in FIG. 17. The skirt 600 can be made from any of the materials disclosed above for skirt 106.

The outer skirt 600 can comprise one or more pleat portions 614. For example, when the skirt 600 is coupled to the frame 102 by the stitches 604, two pleats portions 614 are defined with each pleat portion 614 including at least part of a radially inner layer 610 and at least part of a radially outer layer 612. Specifically, in the illustrated example, the skirt 600 comprises a first (or inflow) pleat portion 614a extending axially from the frame attachment region 605 towards the inflow edge 606 and a second (or outflow) pleat portion 614b extending axially from frame attachment region 605 towards the outflow edge 608. The skirt 600 can define a different number of pleat portions 614, for example, based on the location of the frame attachment region 605, the placement of the stitches 604 and/or number of stitches 604, etc.

The skirt 600 can be configured to protrude radially outward from the frame 102. For example, the pleat portions 614 can be configured to protrude radially outward from the outer surface of the frame 102. In some instances, as shown, the skirt 600 can have one frame attachment region 605 and the stitches 604 can be the only location where the pleat portions 614 are coupled to the frame 102. In this way, the inflow and outflow edges 606, 608 of the pleat portions 614 are free to protrude outward from the frame 102.

The skirt 600 can generally comprise a tubular shape, with an internal space defined between the inner layer 610 and the outer layer 612. In some examples, as depicted in FIG. 17, the skirt 600 includes a spacer 618 positioned within the internal space of the tube-shaped skirt 600. For example, the spacer 618 can be disposed between the inner layer 610 and the outer layer 612. The spacer 618 can be made from any of the materials disclosed above for spacer 318.

Spacers 618 can be disposed within each of the pleat portions 614a, 614b. For example, as depicted in FIG. 17, the skirt 600 can include two spacers 618, with one positioned axially towards the inflow edge 606 relative to the frame attachment region 605 and the other positioned axially towards the outflow edge 608 relative to the frame attachment region 605. In some examples, as shown, the skirt 600 does not include a spacer 618 disposed within the frame attachment region 605, which can enable the skirt 600 to be smaller in a radial direction at the frame attachment region 605 than at the inflow and outflow edges 606, 608 of the skirt. In other examples, the spacer 618 can be positioned within the opening of the skirt 600 at the frame attachment region 605, for example, such that the spacer 618 extends from the inflow edge 606 to the outflow edge 608. In these examples, the radial size of the skirt 600 can be generally uniform in the axial direction.

As introduced above, the skirt 600 can comprise a generally smooth and/or continuous outer surface that does not include folds and/or overlapping edges. In some examples, the shape of the outer surface of the skirt 600 corresponds to the shape of the spacers 618 disposed therein. For example, the spacers 618 can comprise a general smooth, continuous outer surface that is adjacent to an inner surface of the skirt 600 and supports the shape of the skirt 600.

The skirt 600 can be formed by folding a piece of material into the tubular shape. Circumferential (or lengthwise) edges of the skirt 600 can be folded towards each other, for example, towards the frame attachment region 605 of the skirt 600. In some examples, folding the skirt 600 results in one of the layers (for example, inner layer 610) including both edges of the skirt 600. In some instances, as depicted, these edges can be axially spaced apart, such that the layer 610, for example, is discontinuous in the axial direction. For example, the axially spaced-apart edges included in layer 610 can be coupled to the other layer 612, to form the two pleat portions 614a, 614b. For example, the edges can be spaced apart about the frame attachment region 605, such that the frame attachment region 605 includes only one layer (layer 612 in this example). In some instances (see FIG. 5, for example), these edges can be aligned axially (or overlapped axially) and coupled together, such that the layer is continuous in the axial direction.

In some examples, the inner and outer layers 610, 612 can be coupled together with stitches 620 on either side of the frame attachment region 605 towards the inflow and outflow edges 606, 608, respectively. In some instances, as depicted, separate sutures can be used to couple the inner and outer layers 610, 612 together and to couple the skirt 600 to the frame 102. In other instances, the same stitches (e.g., stitches 604) can be used to couple the inner and outer layers 610, 612 together as well as to the frame 102.

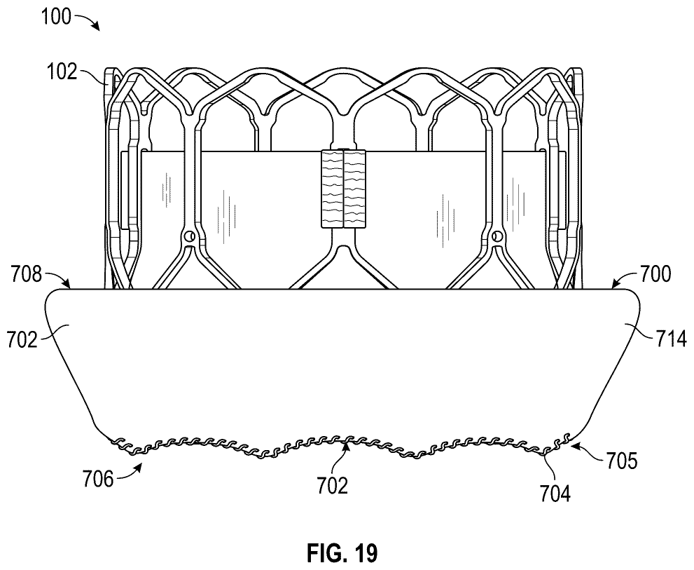

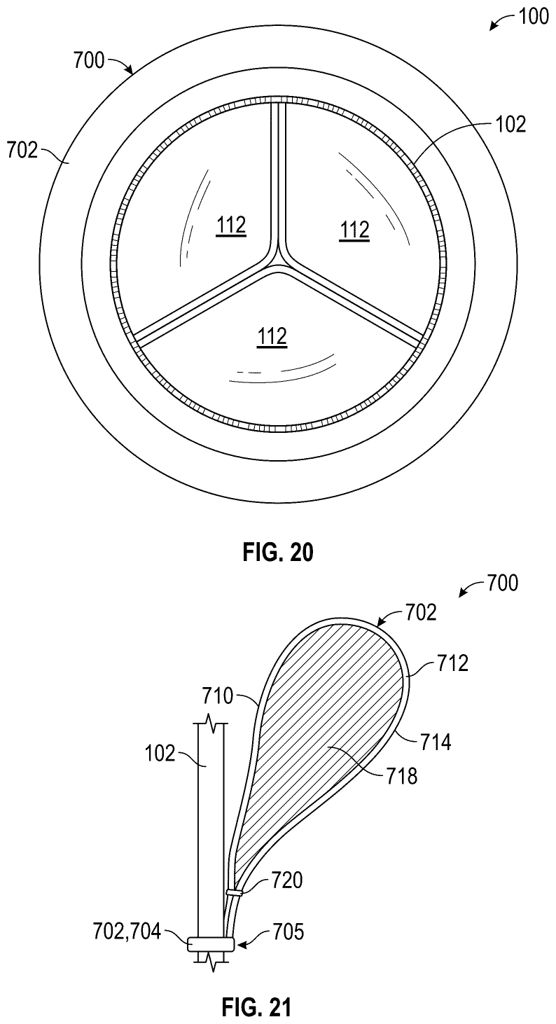

FIGS. 19-21 illustrate another example of an outer skirt, or sealing member, 700 for a prosthetic heart valve. The outer skirt 700 is similar to skirt 400 but does not include longitudinal edges 416, for example. Rather than having overlapping pleats and/or discrete tube segments to define longitudinal edges, the outer skirt 700 is smooth and continuous in the circumferential direction. While shown as coupled to prosthetic heart valve 100, the outer skirt 700 can be used in a mechanically expandable prosthetic valve, other balloon-expandable prosthetic valves (e.g., other than prosthetic heart valve 100 of FIG. 1), and/or a self-expandable prosthetic valve.

The skirt 700 can be coupled to the frame 102 with one or more sutures 702 forming a plurality of stitches 704. For example, the stitches 704 can extend through a frame attachment region 705 of the skirt 700 and around one or more struts 116 of the frame 102. The stitches 704 can extend in a circumferential direction around the skirt 700. As shown in FIG. 19, the frame attachment region 705 is disposed at an inflow edge 706 of the skirt 700. The skirt 700 can comprise a plurality of layers including, for example, an inner layer 710 and an outer layer 712, as shown in FIG. 21. The skirt 700 can be made from any of the materials disclosed above for skirt 106.

As shown, the outer skirt 700 can comprise a pleat portion 714 (also referred to as a “loop portion”) defined by the inner layer 710 and the outer layer 712 and can extend axially from the frame attachment region 705 towards outflow edge 708.

The skirt 700 can be configured to protrude radially outward from the frame 102. For example, the pleat portion 714 can be configured to protrude radially outward from the outer surface of the frame 102. In some instances, as shown, the skirt 700 can have one frame attachment region 705 and the stitches 704 can be the only location where the pleat portion 714 is coupled to the frame 102. In this way, the outflow edge 708 of the pleat portion 714 is free to protrude outward from the frame 102.

The skirt 700 can generally comprise a tubular shape, with an internal space defined between the inner layer 710 and the outer layer 712. In some examples, as depicted in FIG. 21, the skirt 700 includes a spacer 718 positioned within the internal space of the tube-shaped skirt 700. For example, the spacer 718 can be disposed between the inner layer 710 and the outer layer 712. The spacer 718 can be made from any of the materials disclosed above for spacer 318.

As introduced above, the skirt 700 can comprise a generally smooth and/or continuous outer surface that does not include folds and/or overlapping edges. In some examples, the shape of the outer surface of the skirt 700 corresponds to the shape of the spacer 718 disposed therein. For example, the spacer 718 can comprise a general smooth, continuous outer surface that is adjacent to an inner surface of the skirt 700 and supports the shape of the skirt 700.

The skirt 700 can be formed by folding a piece of material into the tubular shape. Circumferential (or lengthwise) edges of the layers 710, 712 can be folded towards each other, for example, towards the frame attachment region 705 of the skirt 700. The layers 710, 712 can be coupled with both edges coupled together and/or with one edge coupled to the opposite layer (either layer 710 or 712) (for example, such that the edges are axially offset). In some examples, the inner and outer layers 710, 712 can be coupled together with stitches 720 adjacent to the frame attachment region 705, for example, positioned on a side of the region 705 towards the outflow edge 708. In other instances, the same stitches (e.g., stitches 704) can be used to couple the inner and outer layers 710, 712 together as well as to the frame 102.

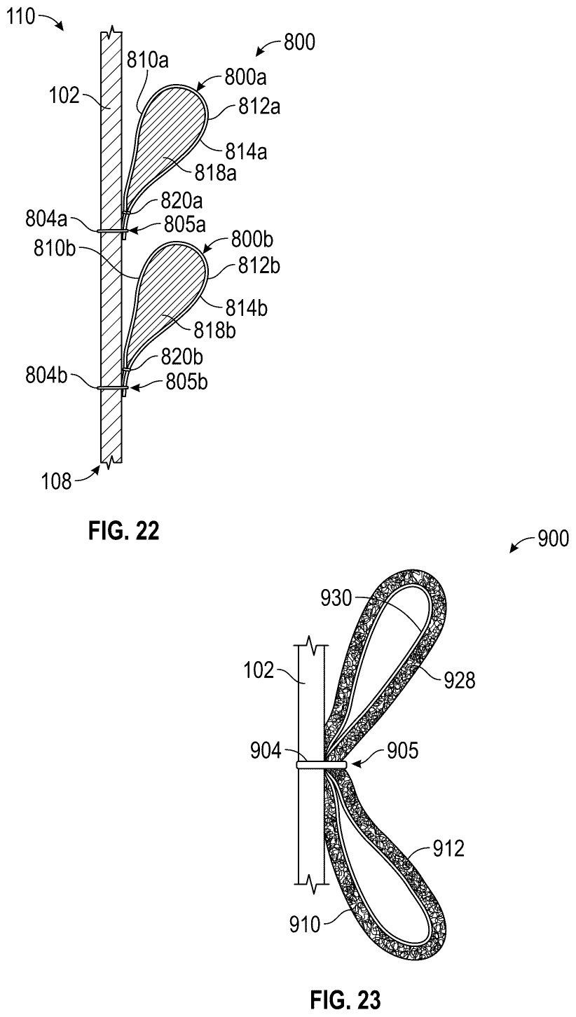

In some examples, as shown in FIG. 22, an outer skirt assembly, or sealing assembly, 800 can comprise two or more skirts coupled to the frame 102. For example, a first skirt, or sealing member, 800a can be positioned towards an outflow end 110 of the frame 102 and a second skirt, or sealing member, 800b can be positioned towards an inflow end 108 of the frame 102. In this way, the skirt assembly 800 can comprise two or more discrete pleat portions 814a, 814b coupled to the frame 102 that are separated in the axial direction. In the illustrated example, the frame attachment region 805a, 805b of each skirt 800a, 800b is positioned towards the inflow end of the frame 102 and the free ends of the pleat portions 814a, 814b are positioned towards the outflow end 110 of the frame 102. In other examples, one or more of the frame attachment regions 805 can be positioned towards the outflow end 110. As shown, each of the pleat portions 814a, 814b can optionally include a spacer 818a, 818b positioned in an internal space between the inner layer 810a, 810b and the outer layer 812a, 812b, respectively. Further, each skirt 800a, 800b can include longitudinal edges (similar to skirts 300, 400, 500, etc.) or be smooth in the circumferential direction (similar to skirts 600, 700, etc.). In some examples, each skirt 800a, 800b can be any of the skirts described herein. The skirt 800 can be made from any of the materials disclosed above for skirt 106.

Optionally, any of the skirts described herein can include a layer of textured or pile material in addition to the inner and outer layers or as one or both of the inner and outer layers. For example, the textured or pile material can include tufts or loops of fibers that extend away from a base of the material. Additional details and examples of materials for sealing members that include pile layers can be found in U.S. Publication No. 2019/0365530, which is incorporated by reference herein.

FIG. 23 illustrates an example of an outer skirt, or sealing member, 900 coupled to a frame 102 that includes a layer 928 of pile material coupled to a base layer 930 of non-pile material. The non-pile material can be made from any of the materials disclosed above for skirt 106. In some examples, as depicted, the pile and base layers 928, 930 together can form an inner layer 910 and an outer layer 912 of the skirt 900. Specifically, layers 928, 930 can be folded over such that the inner layer 910 comprises both the pile and base layers 928, 930 and the outer layer 912 comprises both the pile and base layers 928, 930. In this way, the pile layer 928 defines an outer surface of the skirt 900 to engage with an implantation site. In other examples, the outer layer 912 can comprise only the pile layer 928, rather than the pile layer 928 and the base layer 930. For example, in some instances, a skirt can include a pile layer as the outer layer and a non-pile layer as the inner layer.

In some examples, as shown in FIG. 23, stitches 904 pass through the pile layer 928 and the non-pile layer 930 at the frame attachment region 905 to couple the skirt 900 to the frame 102. In some examples, not shown, the frame attachment region 905 does not include a pile layer 928 and only includes, for example, the non-pile layer 930. This can provide a thinner region for stitches 904 to pass through, for example, without passing through a thicker region that includes both layers 928, 930.

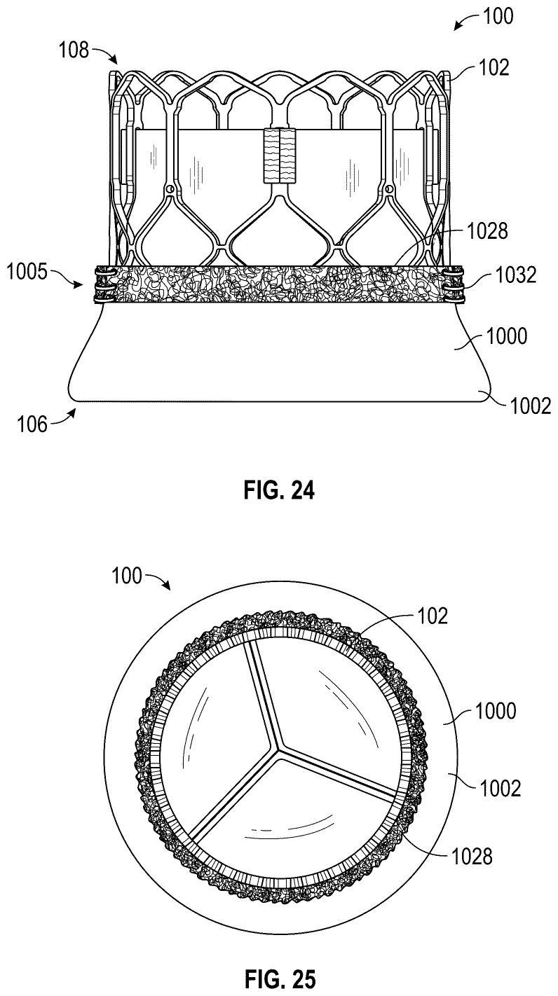

In some examples, a sealing member 1000 for a prosthetic heart valve can include a pile layer coupled to the skirt in other configurations. For example, as shown in FIGS. 24-26, a pile layer 1028 can be coupled to a frame attachment region 1005 of a skirt 1002 and disposed radially outward of the skirt 1002. The skirt 1002 can include any of the skirts described herein. In the illustrated example, the skirt 1002 is similar to the skirt 700 (FIGS. 19-21) but flipped in the axial direction relative to the frame 102. For example, instead of having the free end of the skirt positioned towards the outflow end 108 of the frame 102 and the frame attachment region positioned at the inflow end 108 of the frame (similar to skirt 700), the free end of the skirt 1002 is positioned towards the inflow end 108 of the frame 102 and the frame attachment region 1005 towards the outflow end 110. While shown as coupled to prosthetic heart valve 100, the outer skirt 1002 can be used in a mechanically expandable prosthetic valve, other balloon-expandable prosthetic valves (e.g., other than prosthetic heart valve 100 of FIG. 1), and/or a self-expandable prosthetic valve.

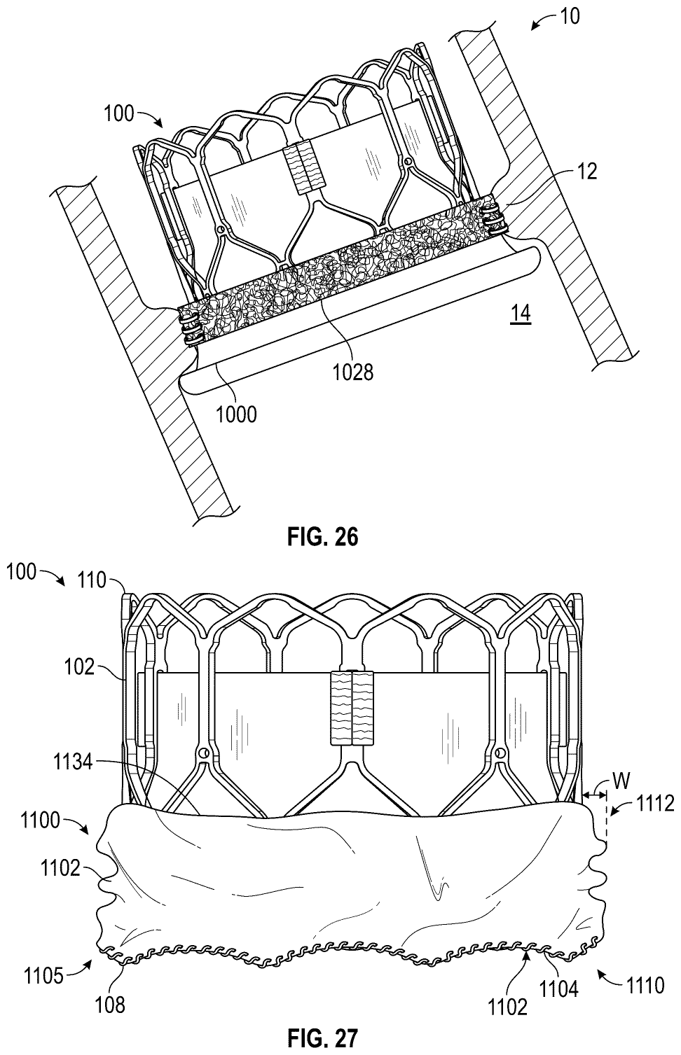

The addition of the pile layer 1028 on an outer surface of the skirt 1002, for example, at the frame attachment region 1005 can increase the texture at this region and/or improve the seal created between the prosthetic heart valve including the sealing member 1000 when positioned at a target implant location, such as within a native aortic annulus 12 of a heart 10 (FIG. 26). For example, the pile layer 1028 can engage with the annulus 12 (shown schematically without the native leaflets for purposes of illustration) and the sealing member 1000 can form a seal with the inflow portion of the annulus 12 and can extend slightly into the left ventricle 14, as depicted.

As shown in FIG. 24, the pile layer 1028 can be coupled to the skirt 1002 and/or to the frame 102 with sutures that form stitches 1032. While shown as coupled at multiple discrete points circumferentially spaced apart around the pile layer 1028, it should be appreciated that the pile layer 1028 can be coupled to the skirt 1002 and/or the frame 102 in other manners.

As introduced above, prosthetic valves (for example, prosthetic valve 100, etc.) can be crimped on or retained by an implant delivery apparatus in a radially compressed state while being advanced through a patient's vasculature on the delivery apparatus. To maintain a low crimp profile, in some examples, a force can be applied to an outer skirt of the prosthetic valve to stretch the outer skirt in an axial direction relative to a frame of the prosthetic valve, as the prosthetic valve is advanced through the patient's vasculature. After the prosthetic valve is advanced to a desired implantation location, the force can be removed which can allow the outer skirt to expand or bulge outwardly in the radial direction.

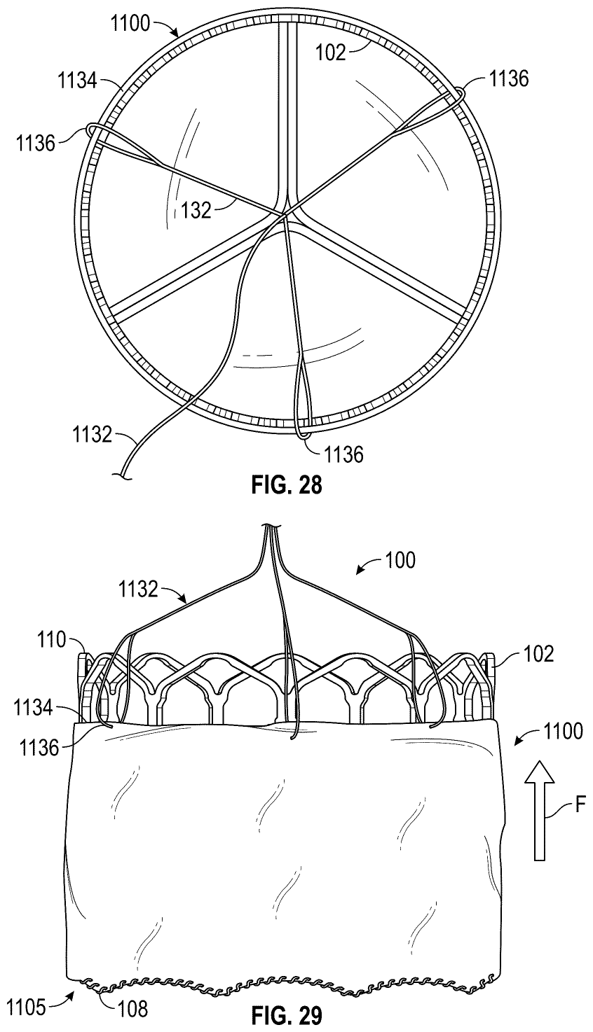

FIG. 27-32 show one example of a stretchable outer skirt, or sealing member, 1100 for a prosthetic device, such as the prosthetic heart valve 100 of FIG. 1, in an unstretched configuration. As described in further detail below, a stretching mechanism 1132 (sutures shown in FIGS. 28-32) can be releasably coupled to the outer skirt 1100 and configured to stretch the outer skirt 1100, for example, when the valve 100 is retained by an implant delivery apparatus in the radially compressed state. While shown as coupled to prosthetic heart valve 100, the outer skirt 1100 can be used in a mechanically expandable prosthetic valve, other balloon-expandable prosthetic valves (e.g., other than prosthetic heart valve 100 of FIG. 1), and/or a self-expandable prosthetic valve.

As shown in FIG. 27, the outer skirt 1100 can be coupled to an outer surface of the frame 102. For example, a frame attachment region 1105 of the skirt 1100 can be positioned at an inflow end 1110 of the skirt 1100 and disposed around the inflow end portion 108 of the frame 102. The skirt 1100 can be coupled to the frame 102 at the frame attachment region 1105 with sutures 1102 forming stitches 1104. At the outflow end 1112 of the skirt 1100, the skirt 1100 is not coupled to the frame 102. Specifically, the skirt 1100 includes a free edge 1134 that is permitted to move relative to the frame 102, including in an axial direction.

The skirt 1100 is configured to be stretched in the axial direction, relative to the frame 102. For example, the skirt 1100 can comprise a material that is configured to elongate in the axial direction when a force is applied to the skirt 1100 and to retract to an unstretched state when the force is removed from and/or not being applied to the skirt 1100. For example, the skirt 1100 can comprise any of the materials described above, including stretchable tissues, stretchable polymers, stretchable fabrics, or the like. In some instances, the skirt 1100 can include strands of elastic fibers or yarns that are oriented in at least the axial direction. Additional details and examples of stretchable materials for sealing members can be found in U.S. Publication No. 2019/0365530, which is incorporated by reference herein.

The skirt 1100 is shown in the unstretched state in FIG. 27. In this state, the skirt 1100 is bunched up and/or consolidated around the frame 102 towards the inflow end 108. In some examples, the free edge 1134 of the skirt 1100 can be drawn towards the frame attachment region 1105 by stretchable strands of the skirt 1100, such as by elastic fibers oriented in the axial direction. As shown, in the unstretched state, the skirt 1100 extends outward and away from the frame 102 in the radial direction by a distance W. In some examples, the distance W can be, for example, in the range of about 5 mm to about 12 mm. This allows the skirt 1100 to conform to the shape of an implantation site, including any irregularities, and form a seal with native anatomy of the implantation site.

As introduced above, a stretching mechanism 1132 can be releasably coupled to the outer skirt 1100 and used to stretch the skirt 1100 in the axial direction relative to the frame 102 by applying a force F. For example, the stretching mechanism 1132 can include sutures 1136 (three shown in FIGS. 28-32) releasably coupled to the free edge 1134 of the skirt 1100. Each suture 1136 can be stitched or looped through the free edge 1134 of the skirt 1100. Specifically, an end of the suture 1136 can pass through the skirt 1100 in the radial direction and be pulled through the skirt 1100 until both ends of the suture 1136 are aligned to form a loop (also referred to as “suture loops 1136”). The attachment point of each suture loop 1136 with the skirt 1100 can be equally spaced apart around the skirt 1100 in the circumferential direction (for example, sixty degrees apart, etc.). While three suture loops 1136 are shown, a greater or lesser number of suture loops 1136 can be used to impart a force F on the skirt 1100 in the axial direction.

FIG. 29 shows the frame 102 of the prosthetic valve 100 in a radially expanded state, with the outer skirt 1100 stretched in the axial direction by the stretching mechanism 1132. Specifically, the suture loops 1136 apply an axially directed force F to the free edge 1134 of the skirt 1100 to pull the free edge 1134 towards the outflow end 110 of the prosthetic valve 100. As shown in FIG. 29, the application of the force F draws the skirt 1100 closer to the frame 102 in the radial direction, such that the skirt 1100 does not extend as far outward from the frame 102 in the radial direction as compared to when force F is not applied (FIG. 27). In some examples, the inner surface of the skirt 1100 can abut the outer surface of the frame 102 when the force F is applied (FIG. 29) and a gap can be created between the inner surface of the skirt 1100 and the outer surface of the frame 102 when the force F is not applied (FIG. 27).

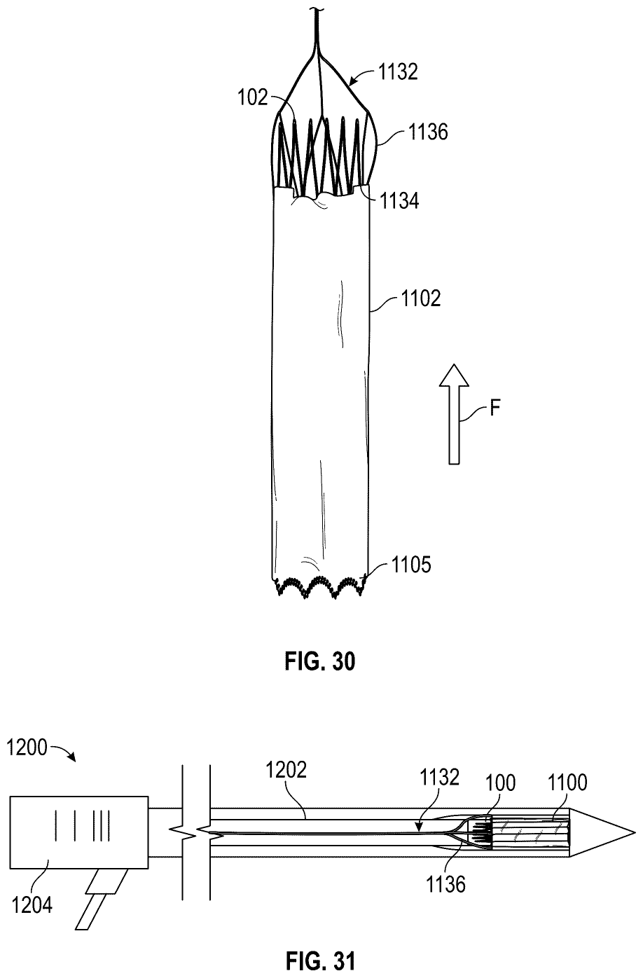

FIG. 30 illustrates the prosthetic valve 100 and the outer skirt 1100 in a delivery configuration. Specifically, the prosthetic valve 100 is in a radially compressed state, with the outer skirt 1100 stretched in the axial direction by the force F applied by the stretching mechanism 1132. As shown in FIG. 31, the prosthetic valve 100 with the outer skirt 1100 can be positioned on a shaft 1202 of a delivery apparatus 1200 while in the delivery configuration. An optional sheath or capsule can extend over the prosthetic valve. In this configuration, the prosthetic valve 100 is positioned towards the distal end of the delivery shaft 1202 and the stretching mechanism 1132 extends proximally from the outer skirt 1100 towards a handle 1204 of the delivery apparatus 1200. Specifically, both ends of each suture loop 1136 are retained within a proximal portion of the delivery apparatus 1200, such as the handle 1204, and can be coupled to a mechanism that maintains the tension of the suture loops 1136 during delivery.

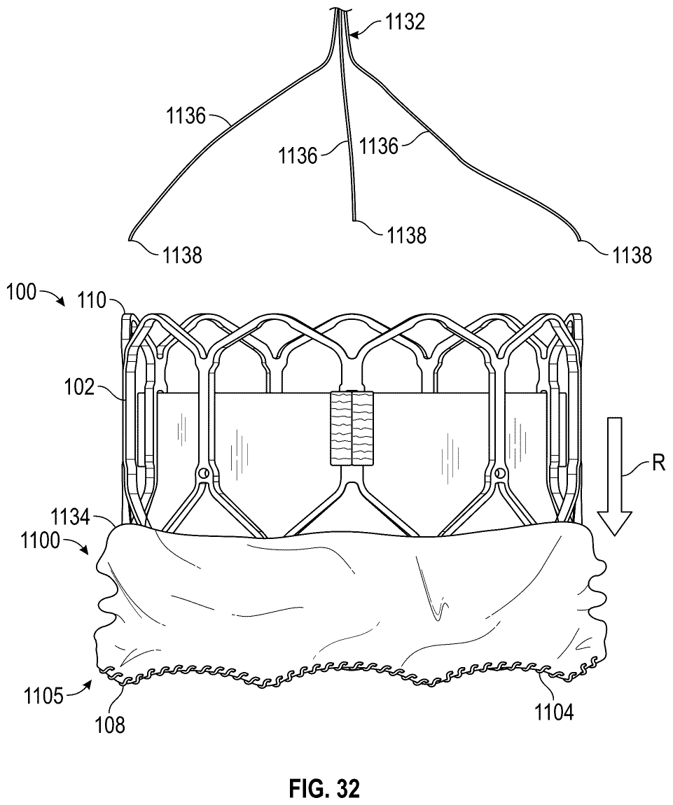

To implant the prosthetic valve 100 and allow the outer skirt 1100 to retract R to the implantation configuration shown in FIG. 32, the stretching mechanism 1132 is detached from the outer skirt 1100. For example, one end of each suture 1136 can be pulled proximally relative to the outer skirt 1100. As one end of each suture 1136 is pulled, the suture 1136 no longer forms a loop and is permitted to slide relative to the skirt 1100 and eventually detach from the skirt 1100. Specifically, the end 1138 of the suture 1136 that is not being pulled is the last portion of the stretching mechanism 1132 to detach from the skirt 1100.

Once the stretching mechanism 1132 is detached from the skirt 1100, the skirt 1100 retracts in the axial direction, towards the frame attachment region 1105. For example, stretchable fibers in the skirt 1100 which were tensioned or stretched by the stretching mechanism 1132 can be unstretched and return to an unbiased position. In this way, the skirt 1100 can be permitted to expand outwardly in the radial direction relative to the frame 102 when the force F is not applied to the skirt 1100 and the stretching mechanism 1132 is detached from the outer skirt 1100. The stretching mechanism 1132 can be detached from the outer skirt 1100 prior to radially expanding the frame 102, after expanding the frame 102, or during radial expansion of the frame 102. The outer skirt 1100 can be released from the stretching mechanism 1132 after the prosthetic valve is radially expanded (FIG. 29), such as by inflating a balloon or releasing the prosthetic valve from a capsule to allow the prosthetic valve to self-expand. Alternatively, the outer skirt 1100 can be released from the stretching mechanism 1132 while the prosthetic valve is still in the radially compressed state.

While the stretching mechanism 1132 of the illustrated example includes sutures 1136, it should be appreciated that other mechanisms can be used to releasably couple to the free edge 1134 of the skirt 1100 and impart an axial force to the skirt 1100 in the delivery configuration. For example, releasable clamps or other gripping devices that engage with the free edge 1134 without extending through the skirt 1100 can be used in some examples.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand) Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) are introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum) into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic valve comprising: a radially expandable frame; a valvular structure disposed within the frame; and a sealing member disposed around an outer surface of the frame, the sealing member having an inflow end, an outflow end, and a frame attachment region positioned axially between the inflow and outflow ends, wherein the sealing member comprises an inner layer and an outer layer, wherein the sealing member is coupled to the frame at the frame attachment region.

Example 2. The prosthetic valve of any example herein, particularly example 1, wherein the inflow and outflow ends of the sealing member are moveable in the radial direction relative to the frame.

Example 3. The prosthetic valve of any example herein, particularly either example 1 or example 2, wherein at least one of the inner layer and the outer layer is coupled to the frame with stitches.

Example 4. The prosthetic valve of any example herein, particularly any one of examples 1-3, wherein the inner layer and the outer layer are both coupled to the frame with the stitches.

Example 5. The prosthetic valve of any example herein, particularly any one of examples 1-4, further comprising at least one spacer disposed within the sealing member between the inner layer and the outer layer.

Example 6. The prosthetic valve of any example herein, particularly example 5, wherein the spacer comprises a compressible material.

Example 7. The prosthetic valve of any example herein, particularly any one of examples 1-6, wherein the sealing member comprises axially-extending edges.

Example 8. The prosthetic valve of any example herein, particularly example 7, wherein the sealing member comprises a plurality of axial folds defining the edges.

Example 9. The prosthetic valve of any example herein, particularly example 7, where the sealing member comprises a plurality of discrete segments defining the edges, wherein each segment is partially overlapped in the radial direction with an adjacent segment.

Example 10. The prosthetic valve of any example herein, particularly example 9, wherein each segment defines one of the edges.

Example 11. The prosthetic valve of any example herein, particularly any one of examples 1-10, wherein the sealing member comprises a material that is thinner at the frame attachment region than at the inflow and outflow ends of the sealing member.

Example 12. The prosthetic valve of any example herein, particularly any one of examples 1-11, wherein an outer surface of the sealing member comprises a pile texture.

Example 13. The prosthetic valve of any example herein, particularly any one of examples 1-12, further comprising a textured layer disposed radially outward of the sealing member at the frame attachment region.

Example 14. A prosthetic valve comprising: a radially expandable frame; a valvular structure disposed within the frame; and a sealing member disposed around an outer surface of the frame, the sealing member comprising at least one pleat portion having an inner layer and an outer layer, wherein the pleat portion includes a fixed end coupled to the frame and a free end that is moveable relative to the frame.

Example 15. A prosthetic valve comprising: a radially expandable frame; a valvular structure disposed within the frame; and a sealing member disposed around an outer surface of the frame and comprising a plurality of axially-extending pleats.

Example 16. The prosthetic valve of any example herein, particularly example 15, wherein the pleats overlap each other in a circumferential direction.

Example 17. The prosthetic valve of any example herein, particularly example 15 or 16, wherein the sealing member comprises an inner layer and an outer layer forming the axially extending pleats.

Example 18. The prosthetic valve of any example herein, particularly example 17, wherein the sealing member comprises at least one spacer disposed between the inner layer or the outer layer.

Example 19. The prosthetic valve of any example herein, particularly examples 17-18, wherein the inner layer and the outer layer are secured with a plurality of stitches that form a circumferentially extending stitch line.

Example 20. The prosthetic valve of any example herein, particularly example 19, wherein the stitch line is located axially between an inflow end and an outflow end of the sealing member.

Example 21. The prosthetic valve of any example herein, particularly any of examples 15-20, wherein the sealing member comprises a plurality of skirt segments defining one or more of the axially-extending edges.

Example 22. The prosthetic valve of any example herein, particularly example 21, wherein each skirt segment overlaps an adjacent skirt segment in a circumferential direction.

Example 23. A prosthetic valve comprising: a radially expandable frame having an inflow end portion and an outflow end portion; a valvular structure disposed within the frame; and a sealing member disposed around an outer surface of the frame, the sealing member comprising a fixed end coupled to the frame and a free end moveable relative to the frame, the sealing member configured to be stretched in an axial direction by a force applied in an axial direction to the free end.

Example 24. The prosthetic valve of any example herein, particularly example 23, wherein the fixed end is coupled to the inflow end portion of the frame.

Example 25. The prosthetic valve of any example herein, particularly either example 23 or example 24, further comprising a suture removably coupled to the free end of the sealing member, wherein the suture is configured to apply the force in the axial direction.

Example 26. The prosthetic valve of any example herein, particularly any one of examples 23-25, wherein the sealing member is configured to retract in the axial direction when the force is removed from the free end.

Example 27. The prosthetic valve of any example herein, particularly any one of examples 23-26, wherein a thickness of the sealing member is configured to increase in the radial direction when the force is removed from the free end.

Example 28. The prosthetic valve of any example, herein, particularly any one of examples 1-27, wherein the prosthetic valve is sterilized.

The features described herein with regard to any example can be combined with other features described in any one or more of the other examples, unless otherwise stated. For example, any one or more of the features of one prosthetic valve can be combined with any one or more features of another prosthetic valve. As another example, any one or more features of one skirt assembly can be combined with any one or more features of another skirt assembly.

In view of the many possible ways in which the principles of the disclosure may be applied, it should be recognized that the illustrations depict examples of the disclosed technology and should not be taken as limiting the scope of the disclosure nor the claims. Rather, the scope of the claimed subject matter is defined by the following claims and their equivalents.

We claim:

1. A prosthetic valve comprising:
a radially expandable frame;
a valvular structure disposed within the frame; and
a sealing member disposed around an outer surface of the frame, the sealing member having an inflow end, an outflow end, and a frame attachment region positioned axially between the inflow and outflow ends, wherein the sealing member comprises an inner layer and an outer layer, wherein the sealing member is coupled to the frame at the frame attachment region.

2. The prosthetic valve of claim 1, wherein the inflow and outflow ends of the sealing member are moveable in the radial direction relative to the frame.

3. The prosthetic valve of claim 1, wherein at least one of the inner layer and the outer layer is coupled to the frame with stitches.

4. The prosthetic valve of claim 1, wherein the inner layer and the outer layer are both coupled to the frame with the stitches.

5. The prosthetic valve of claim 1, further comprising at least one spacer disposed within the sealing member between the inner layer and the outer layer.

6. The prosthetic valve of claim 5, wherein the spacer comprises a compressible material.

7. The prosthetic valve of claim 1, wherein the sealing member comprises axially-extending edges.

8. The prosthetic valve of claim 7, wherein the sealing member comprises a plurality of axial folds defining the edges.

9. The prosthetic valve of claim 7, where the sealing member comprises a plurality of discrete segments defining the edges, wherein each segment is partially overlapped in the radial direction with an adjacent segment.

10. The prosthetic valve of claim 9, wherein each segment defines one of the edges.

11. The prosthetic valve of claim 1, wherein the sealing member comprises a material that is thinner at the frame attachment region than at the inflow and outflow ends of the sealing member.

12. The prosthetic valve of claim 1, wherein an outer surface of the sealing member comprises a pile texture.

13. The prosthetic valve of claim 1, further comprising a textured layer disposed radially outward of the sealing member at the frame attachment region.

14. A prosthetic valve comprising:
a radially expandable frame;
a valvular structure disposed within the frame; and
a sealing member disposed around an outer surface of the frame, the sealing member comprising at least one pleat portion having an inner layer and an outer layer, wherein the pleat portion includes a fixed end coupled to the frame and a free end that is moveable relative to the frame.

15. A prosthetic valve comprising:
a radially expandable frame;
a valvular structure disposed within the frame; and
a sealing member disposed around an outer surface of the frame and comprising a plurality of axially-extending pleats.

16. The prosthetic valve of claim 15, wherein the pleats overlap each other in a circumferential direction.

17. The prosthetic valve of claim 15, wherein sealing member comprises an inner layer and an outer layer forming the axially-extending pleats.

18. The prosthetic valve of claim 17, wherein the inner layer and the outer layer are secured with a plurality of stitches that form a circumferentially extending stitch line.

19. The prosthetic valve of claim 18, wherein the stitch line is located axially between an inflow end and an outflow end of the sealing member.

20. The prosthetic valve of claim 15, wherein the sealing member comprises a plurality of skirt segments defining one or more of the axially-extending edges.
